(19) United States
(12) Patent Application Publication
(10) Pub. No.: US 2007/0123508 A1
(43) Pub. Date: May 31, 2007

(54) PAR2-modulating compounds and their use

(76) Inventors: Roger Olsson, Bunkeflostrand (SE); Jimmi Gerner Seitzberg, Malmo (SE)

Correspondence Address: SQUIRE, SANDERS & DEMPSEY L.L.P., 1 MARITIME PLAZA, SUITE 300, SAN FRANCISCO, CA, 94111, US

(21) Appl. No.: 11/436,130

(22) Filed: May 16, 2006

Related U.S. Application Data
(60) Provisional application No. 60/685,125, filed on May 27, 2005.

(51) Int. Cl. A61K 31/55; A61K 31/445; A61K 31/4015; C07D 211/54

(52) U.S. Cl. 514/212.03; 514/317; 514/327; 514/423; 540/529; 546/216; 548/530

(57) ABSTRACT

This invention relates to compounds, their uses for the elucidation of PAR2 activity and their uses for the treatment or prevention of diseases or disorders related to PAR2 activity, wherein the compound has the general chemical structure:

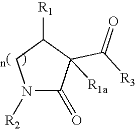

### RELATED APPLICATIONS

This application is related to and claims the benefit of U.S. Provisional Application Ser. No. 60/685,125, filed 27 May 2005.

### FIELD

This invention relates to the fields of organic chemistry, pharmaceutical chemistry, biochemistry, molecular biology and medicine. In particular it relates to compounds that modulate the activity of proteinase-activated receptor-2 (PAR2), to the use of the compounds as tools for the further elucidation of the role of PAR2 in biological systems and to the treatment and prevention of diseases and disorders related to PAR-2

### BACKGROUND

In 1991, Vu, et al. (Cell, 1991, 64:1057-68) first reported a new receptor in the G-protein-coupled superfamily of receptors that was activated by an unexpected and entirely novel mechanism. The new receptor was found to comprise seven transmembrane domains, three intracellular domains, three extracellular loops, an extracellular N-terminus and a C-tail within the cell. The novel activation mechanism involved the serine protease thrombin, which cleaved the receptor at a specific site in the extracellular N-terminus, thus revealing an N-tethered ligand domain that then intramolecularly bound to and activated the receptor.

It was not until 1994 that it was discovered that Vu's receptor was not one of a kind. In that year, Nystead, et al. (J. Proc. Natl. Acad. Sci (USA), 1994, 91:9208-12) reported a second receptor that was activated by a protease. This receptor was cleaved in its extracellular N-terminus by trypsin to similarly expose an N-tethered ligand that intramolecularly bound and activated the receptor. The discovery prompted the establishment of a new family of receptors, coined the proteinase-activated receptors or PARs.

Since the discovery of the second PAR, two more PARs have been discovered giving four in all. They are designated PAR1, PAR2, PAR3 and PAR4. PAR2 is distinguished from the other three in that it is the only one of the group that is activated by trypsin and tryptase. The other three PARs are all activated by thrombin. Since its discovery, PAR2 receptor has been implicated in numerous physiological processes necessitating therapeutic intervention including inflammation, colitis, asthma, pruritis, tear secretion, bone remodeling, vascular tone, ischemia and nociception and in particular, PAR2 is thought to exert a protective effect in the airways, pancreas, GI tract, and in ischemia in the brain and heart. PAR2 is expressed in the cardiovascular system, where it is suggested to play an important role in vascular tone and alterations in vascular function during inflammation with implications in, without limitation, hypertension. It is expressed in the gastrointestinal system, e.g., the small intestine and colon, as well as in the exocrine organs of the digestive tract, i.e., the stomach, pancreas and salivary glands. It is expressed in the myenteric and submucosal nerve plexuses, the signaling of which may alter the regulation of intestinal motility and secretion. Thus, PAR2 may be implicated in gastrointestinal diseases such as, without limitation, inflammatory bowel disease, irritable bowel disease, pancreatitis and gastritis. PAR2 is expressed in the pulmonary system where its role in the modulation of inflammatory processes suggests it as a pharmacological target for pulmonary diseases such as, without limitation, asthma and chronic obstructive pulmonary disease. PAR2 has also been implicated in the pathology of skin diseases such as, without limitation, cutaneous neurogenic inflammation, pruritis, dry skin syndrome and other inflammatory skin diseases. PAR2 is also strongly expressed in human colon adenocarcinoma cells suggesting a role in cancer. Studies have suggested that PAR2 may play a key role in neurogenic inflammation and pain. It has also been implicated in the genesis of visceral pain (see refs. 1-22).

Given the apparent ubiquity of PAR2 and its participation in diverse physiological and pathophysiological processes in various organ systems including, but not limited to, the cardiovascular system, the pulmonary system, the gastrointestinal tract and the skin, it would be extremely valuable to have compounds that are specific agonists and antagonists of PAR2, both as therapeutic agents and as tools in the exploration of this novel receptor.

### SUMMARY

Thus, in one aspect the present invention is related to a compound having the chemical structure 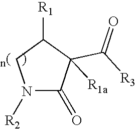
or a pharmaceutically acceptable salt or prodrug thereof, wherein:
n is 1, 2, or 3;
R₁ is selected from the group consisting of hydrogen, alkyl; alkenyl, cycloalkyl, cycloalkenyl, substituted or unsubstituted aryl, substituted or unsubstituted heteroaryl, substituted or unsubstituted heteroalicyclyl, —CN, —C(═Z)R₄, —C(═Z)OR₄, —C(═Z)NR₄R₄ₐ, —N(R₄)—C(═Z)R₄ₐ, —N(R₄)—C(═Z)NR_{4b}R₄ₐ, —OC(═Z)R₄, and —SR₄;
R₁ₐ is selected from the group consisting of hydrogen, alkyl, alkenyl, alkynyl, aryl, halogen, hydroxyl, nitro, amino, sulfonyl, perhaloalkyl, —OR₄, —NR₄R₄ₐ, —CN, —C(═Z)R₄, —C(═Z)OR₄, —C(═Z)NR₄R₄ₐ;
R₂ is selected from the group consisting of hydrogen, alkyl, alkenyl, cycloalkyl, cycloalkenyl, substituted or unsubstituted aryl and unsubstituted or substituted heteroaryl; and,
R₃ is selected from the group consisting of hydrogen, alkyl, alkenyl, cycloalkyl, cycloalkenyl, substituted or unsubstituted aryl, substituted or unsubstituted heteroaryl, substituted or unsubstituted heteroalicyclyl, hydroxy, perhaloalkyl, —OR₄, —NR₄R₄ₐ, —NR₄NR₄ₐR_{4b}, —NR₄N═CR₄ₐR_{4b}, —N(R₄)C(R₄ₐ)═NR_{4b}, —N(R₄)—C(═Z)R₄ₐ, —N(R₄)—C(═Z)NR_{4b}R₄ₐ, and —SR₄, wherein:
   Z is oxygen or sulfur; and,
   R₄, R₄ₐ and R_{4b} are independently selected from the group consisting of: hydrogen, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroaryl, substituted or unsubstituted heteroalicyclyl.

In an aspect of this invention, R₃ is selected from the group consisting of —CH₂R₅, —OR₅ₐ and —NR_{5b}R_{5c}, wherein:
R₅ is selected from the group consisting of hydrogen; alkyl, alkenyl, cycloalkyl, cycloalkenyl, substituted or unsubstituted aryl, substituted or unsubstituted heteroaryl, hydroxy, nitro, amino, halogen, sulfonyl, perhaloalkyl, —OR₄, —NR₄R₄ₐ, —N═CR₄R₄ₐ, —C(R₄)═NR₄ₐ, —CN, —C(═Z)R₄, —C(═Z)OR₄, —C(═Z)NR₄R₄ₐ, —N(R₄)—C(═Z)R₄ₐ, —N(R₄)—C(═Z)NR₄ₐR_{4b}, —OC(═Z)R₄, and —SR₄;
R₅ₐ is selected from the group consisting of hydrogen; alkyl, alkenyl, cycloalkyl, cycloalkenyl, substituted or unsubstituted aryl, substituted or unsubstituted heteroaryl, and,
R_{5b} and R_{5c}, is selected from the group consisting of hydrogen; alkyl, alkenyl, cycloalkyl, cycloalkenyl, substituted or unsubstituted aryl, substituted or unsubstituted heteroaryl, hydroxy, amino, sulphonate, perhaloalkyl, —OR₄, —NR₄R₄ₐ, —N═CR₄R₄ₐ, —C(R₄)═NR₄ₐ, —CN, —C(═Z)R₄, —C(═Z)OR₄, —C(═Z)NR₄R₄ₐ, —N(R₄)—C(═Z)R₄ₐ, —N(R₄)—C(═Z)NR₄ₐR_{4b}, —OC(═Z)R₄, and —SR₄.

In an aspect of this invention, R₃ is selected from the group consisting of: 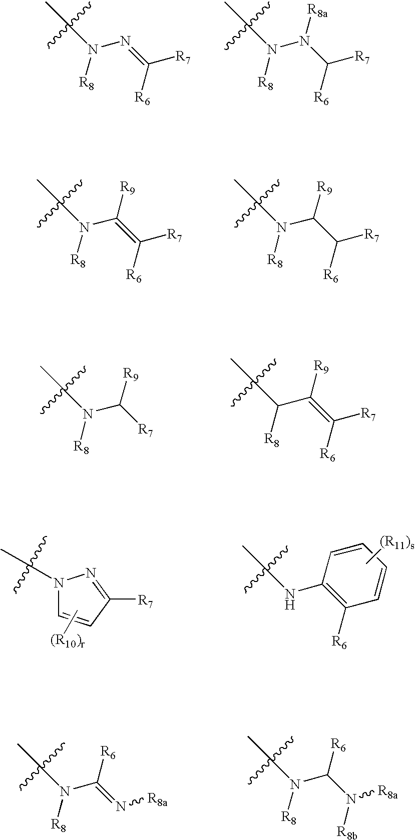
wherein;
R₆, R₇, and R₉ are independently selected from the group consisting of hydrogen, alkyl, alkenyl, cycloalkyl, cycloalkenyl, substituted or unsubstituted aryl, substituted or unsubstituted heteroaryl, substituted or unsubstituted heteroalicyclyl, hydroxy, nitro, amino, halogen, sulfonate, perhaloalkyl, —OR₄, —NR₄R₄ₐ, —CN, —C(═Z)R₄, —C(═Z)OR₄, —C(═Z)NR₄R₄ₐ, —N(R₄)—C(═Z)R₄ₐ, —N(R₄)—C(═Z)NR₄ₐR_{4b}, —OC(═Z)R₄, and —SR₄;
R₈, R₈ₐ and R_{8b} are independently selected from the group consisting of hydrogen, alkyl, alkenyl, cycloalkyl, cycloalkenyl, substituted or unsubstituted aryl; substituted or unsubstituted heteroaryl, substituted or unsubstituted heteroalicyclyl, hydroxy, nitro, amino, halogen, sulfonyl, perhaloalkyl, —OR₄, —NR₄R₄ₐ, —CN, —C(═Z)R₄, —C(═Z)OR₄, —C(═Z)NR₄R₄ₐ, —N(R₄)—C(═Z)R₄ₐ, —N(R₄)—C(═Z)NR₄ₐR_{4b}, —OC(═Z)R₄, and —SR₄;
R₁₀ and R₁₁ are independently selected from the group consisting of hydrogen, alkyl, alkenyl, cycloalkyl, cycloalkenyl, substituted or unsubstituted aryl; substituted or unsubstituted heteroaryl, substituted or unsubstituted heteroalicyclyl, hydroxy, nitro, amino, halogen, sulfonyl, perhaloalkyl, —OR₄, —NR₄R₄ₐ, —CN, —C(═Z)R₄, —C(═Z)OR₄, —C(═Z)NR₄R₄ₐ, —N(R₄)—C(═Z)R₄ₐ, —N(R₄)—C(═Z)NR₄ₐR_{4b}, —OC(═Z)R₄, and —SR₄;
r is 0, 1 or 2; and
s is 0, 1, 2, 3 or 4.

In an aspect of this invention, R₁ is selected from the group consisting of substituted or unsubstituted aryl, substituted or unsubstituted heteroaryl, wherein if substituted, the substituent is one or more independently selected from the group consisting of alkyl, alkenyl, cycloalkyl, cycloalkenyl, substituted or unsubstituted aryl, substituted or unsubstituted heteroaryl, substituted or unsubstituted heteroalicyclyl, hydroxy, nitro, amino, halogen, sulfonate, perhaloalkyl, —OR₄, —NR₄R₄ₐ, —CN, —C(═Z)R₄, —C(═Z)OR₄, —C(═Z)NR₄R₄ₐ, —N(R₄)—C(═Z)R₄ₐ, —N(R_{4b})—C(═Z)NR₄R₄ₐ, —OC(═Z)R₄, and —SR_{4.}

In an aspect of this invention, R₃ is selected from the group consisting of: 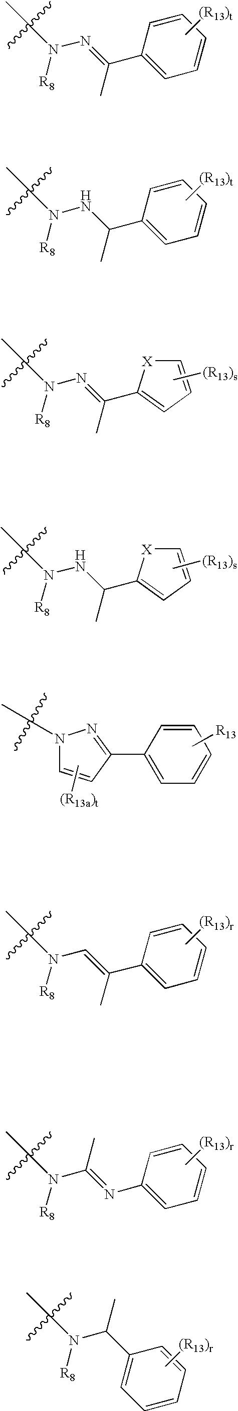
wherein:
R₈ is hydrogen or alkyl;
X is NH, O or S;
   each R₁₃ is independently selected from the group consisting of hydrogen, alkyl, alkenyl, alkynyl, hydroxy, nitro, amino, halogen, sulfonyl, perhaloalkyl, —OR₄, —NR₄R₄ₐ, —CN, —C(═Z)R₄, —C(═Z)OR₄, —C(═Z)NR₄R₄ₐ, —N(R₄)—C(═Z)R₄ₐ, —N(R_{4b})—C(═Z)NR₄R₄ₐ, —OC(═Z)R₄, and —SR4;
r is 0, 1, 2, 3, 4 or 5;
s is 0, 1, 2, 3 or 4; and,
t is 0, 1 or 2.

An aspect of this invention is a method for treating or preventing a disease or disorder related to abnormal PAR2 activity, comprising administering a therapeutically effective amount of a compound of claim 1 to a patient in need thereof.

In an aspect of this invention, the disease or disorder is selected from the group consisting of:
acute or chronic pain;
acute or chronic inflammation;
diseases or disorder of the pulmonary system;
diseases or disorders of the gastrointestinal system;
diseases or disorders of the musculoskeletal system;
diseases or disorders of the central nervous system;
diseases or disorders of the cardiovascular system;
disease or disorders of the renal system;
diseases or disorders of the hepatic system;
diseases of disorders of the eye;
diseases or disorders of the skin;
diseases or disorders of the prostrate;
diseases or disorders of the pancreas;
Sjogren's syndrome; and,
dry mouth.

In an aspect of this invention, the disease or disorder of the pulmonary system is selected from the group consisting of asthma, chronic obstructive pulmonary disease, lung cancer and pneumonitis.

In an aspect of this invention, the disease or disorder of the gastrointestinal system is selected from the group consisting of gastric ulcers, colitis, inflammatory bowel syndrome, Crohn's disease, gastric and intestinal motility, colon cancer, cancer of the stomach, and cancer of the intestine.

In an aspect of this invention, the disease or disorder of the musculoskeletal system is selected from the group consisting of rheumatoid arthritis, osteoporosis and Paget's disease.

In an aspect of this invention, the disease or disorder of the central nervous system is selected from the group consisting of Alzheimer's disease, encephalitis, meningitis, ischemia and stroke.

In an aspect of this invention, the disease or disorder of the cardiovascular system is selected from the group consisting of hypertension, atherosclerosis, angina, congestive heart failure, myocarditus and cardiac ischemia.

In an aspect of this invention, the disease or disorder of the renal system is selected from the group consisting of glomerular kidney disease, kidney cancer and renal failure.

In an aspect of this invention, the disease or disorder of the hepatic system is selected from the group consisting hepatitis and liver cancer.

In an aspect of this invention, the disease or disorder of the eye is selected from the group consisting of glaucoma, retinitis pigmentosa, cataracts, macular degeneration and dry eye.

In an aspect of this invention, the disease or disorder of the skin is selected from the group consisting of dermatitis, psoriasis, pruritis, dermatitis, eczema, seborrhea, wounds, and melanoma.

In an aspect of this invention, the disease or disorder of the pancreatic system is selected from the group-consisting of pancreatitus, pancreatic cancer and diabetes.

In an aspect of this invention, the disease or disorder is dry mouth.

In an aspect of this invention, the disease or disorder is Sjogren's syndrome.

In an aspect of this invention, the disease or disorder is acute or chronic pain.

In an aspect of this invention, the disease or disorder is acute or chronic inflammation.

In an aspect of this invention, the disease of disorder of the prostatic system is selected from the group consisting of benign prostatic hyperplasia and prostatic cancer.

In an aspect of this invention, the disease or disorder of the pancreatic system is selected from the group consisting of pancreatitis, diabetes and pancreatic cancer.

An aspect of this invention is a compound selected from the group consisting of:
2-Oxo-4-phenyl-3-pyrrolidinecarboxylic acid [1-(3-bromo-phenyl)-(E/Z)-ethylidene]-hydrazide (3)
2-Oxo-4-phenyl-3-pyrrolidinecarboxylic acid [1-(5-bromo-thiophen-2-yl)-(E/Z)-ethylidene]-hydrazide (4)
2-Oxo-4-phenyl-3-pyrrolidinecarboxylic acid [1-(thiophen-2-yl)-(E/Z)-ethylidene]-hydrazide (5)
2-Oxo-4-phenyl-3-pyrrolidinecarboxylic acid [1-(4-bromo-thiophen-2-yl)-(E/Z)-ethylidene]-hydrazide (6)
2-Oxo-4-phenyl-3-pyrrolidinecarboxylic acid [1-(5-bromo-pyridine-3-yl)-(E/Z)-ethylidene]-hydrazide (7)
2-Oxo-4-phenyl-3-pyrrolidinecarboxylic acid [1-(4-bromo-phenyl)-(E/Z)-ethylidene]-hydrazide (8)
2-Oxo-4-phenyl-3-pyrrolidinecarboxylic acid [1-(2-bromo-phenyl)-(E/Z)-ethylidene]-hydrazide (9)
2-Oxo-4-phenyl-3-pyrrolidinecarboxylic acid [1-(3-methoxy-phenyl)-(E/Z)-ethylidene]-hydrazide (10)
2-Oxo-4-phenyl-3-pyrrolidinecarboxylic acid [1-(toluen-3-yl)-(E/Z)-ethylidene]-hydrazide (11)
2-Oxo-4-phenyl-3-pyrrolidinecarboxylic acid [1-(3-trifluoromethyl-phenyl)-(E/Z)-ethylidene]-hydrazide (12)
2-Oxo-4-phenyl-3-pyrrolidinecarboxylic acid N′-[1-(3-bromo-phenyl)-(E/Z)-ethyl]-hydrazide (13)
2-oxo-4-phenyl-3-pyrrolidinecarboxylic acid 2-[(E/Z)-1-(3,5-dimethylphenyl)ethylidene]hydrazide (14)
2-oxo-4-phenyl-3-pyrrolidinecarboxylic acid 2-[(1 E/Z)-7-bromo-2,3-dihydro-4-methyl-1H-inden-1-ylidene]hydrazide (15)
2-oxo-4-phenyl-3-pyrrolidinecarboxylic acid 2-[(1 E/Z)-5-(acetylamino)-2,3-dihydro-1H-inden-1-ylidene]hydrazide (16)
2-oxo-4-phenyl-3-pyrrolidinecarboxylic acid 2-[(E/Z)-1-(5-carboxy-2-thienyl)ethylidene]hydrazide (17)
2-oxo-4-phenyl-3-pyrrolidinecarboxylic acid 2-[(E/Z)-1-(4-chlorophenyl)-3-(dimethylamino)propylidene]hydrazide (18)
2-oxo-4-phenyl-3-pyrrolidinecarboxylic acid 2-[(E/Z)-1-(1H-pyrrol-2-yl)ethylidene]hydrazide (19)
2-oxo-N′-[(E/Z)-1-(3-oxo-3,4-dihydro-2H-1,4-benzoxazin-6-yl)ethylidene]-4-phenyl-3-pyrrolidinecarbohydrazide (20)
2-N′-[(4E/Z)-2,3-dihydro-4H-chromen-4-ylidene]-2-oxo-4-phenyl-3-pyrrolidinecarbohydrazide (21)
2-oxo-4-phenyl-3-pyrrolidinecarboxylic acid 2-[(E/Z)-1-(3-fluorophenyl)ethylidene]hydrazide (22)
2-oxo-4-phenyl-3-pyrrolidinecarboxylic acid 2-[(E/Z)-1-(3-chlorophenyl)ethylidene]hydrazide (23)
2-oxo-4-phenyl-3-pyrrolidinecarboxylic acid 2-[(E/Z)-1-(3-cyanophenyl)ethylidene]hydrazide (24)
2-oxo-4-phenyl-3-pyrrolidinecarboxylic acid 2-[(E/Z)-1-(3-hydroxyphenyl)ethylidene]hydrazide (25)
2-oxo-4-phenyl-3-pyrrolidinecarboxylic acid 2-[(E/Z)-1-(3,5-dimethoxyphenyl)ethylidene]hydrazide (26)
2-oxo-4-phenyl-3-pyrrolidinecarboxylic acid 2-[(E/Z)-1-[3,5-bis(trifluoromethyl)phenyl]ethylidene]hydrazide (27)
2-oxo-4-phenyl-3-pyrrolidinecarboxylic acid 2-[(E/Z)-1-(3,5-difluorophenyl)ethylidene]hydrazide (28)
2-oxo-4-phenyl-3-pyrrolidinecarboxylic acid 2-[(E/Z)-1-(3,4-dimethylphenyl)ethylidene]hydrazide (29)
2-oxo-4-phenyl-3-pyrrolidinecarboxylic acid 2-[(E/Z)-1-(1,3-benzodioxol-5-yl)ethylidene]hydrazide (30)
2-oxo-4-phenyl-3-pyrrolidinecarboxylic acid 2-[(E/Z)-1-(2,3-dihydro-1,4-benzodioxin-6-yl)ethylidene]hydrazide (31)
2-oxo-4-phenyl-3-pyrrolidinecarboxylic acid 2-[(E/Z)-1-(2,3-dihydro-1,4-benzodioxin-6-yl)ethylidene]hydrazide (32)
2-oxo-4-phenyl-3-pyrrolidinecarboxylic acid 2-[(E/Z)-1-(2,3-dihydro-2-oxo-6-benzothiazolyl)ethylidene]hydrazide (33)
2-oxo-4-phenyl-3-pyrrolidinecarboxylic acid 2-[(E/Z)-1-(2,5-dichloro-3-thienyl)ethylidene]hydrazide (34)
2-oxo-4-phenyl-3-pyrrolidinecarboxylic acid 2-[(E/Z)-1-(2,5-dimethyl-3-thienyl)ethylidene]hydrazide (35)
2-oxo-4-phenyl-3-pyrrolidinecarboxylic acid 2-[(E/Z)-1-(2,5-dimethyl-3-furanyl)ethylidene]hydrazide (36)
2-oxo-4-phenyl-3-pyrrolidinecarboxylic acid 2-[(E/Z)-1-(2,5-dimethyl-3-furanyl)ethylidene]hydrazide (37)
2-oxo-4-phenyl-3-pyrrolidinecarboxylic acid 2-[(E/Z)-1-(5-chloro-2-thienyl)ethylidene]hydrazide (38)
2-oxo-4-phenyl-3-pyrrolidinecarboxylic acid 2-[(E/Z)-1-(5-methyl-2-thienyl)ethylidene]hydrazide (39)
2-oxo-4-phenyl-3-pyrrolidinecarboxylic acid 2-[(E/Z)-1-(5-cyano-2-thienyl)ethylidene]hydrazide (40)
2-oxo-4-phenyl-3-pyrrolidinecarboxylic acid 2-[(E/Z)-1-(5-bromo-2-hydroxyphenyl)ethylidene]hydrazide (41)
2-oxo-4-phenyl-3-pyrrolidinecarboxylic acid 2-[(E/Z)-1-(4-methyl-2-thienyl)ethylidene]hydrazide (42)
2-oxo-4-phenyl-3-pyrrolidinecarboxylic acid 2-[(1 E/Z)-2,3-dihydro-5-methoxy-1H-inden-1-ylidene]hydrazide (43)
2-oxo-4-phenyl-3-pyrrolidinecarboxylic acid 2-[(1 E/Z)-2,3-dihydro-5,6-dimethoxy-1H-inden-1-ylidene]hydrazide (44)
2-oxo-4-phenyl-3-pyrrolidinecarboxylic acid 2-[(1 E/Z)-5-fluoro-2,3-dihydro-1H-inden-1-ylidene]hydrazide (45)
2-oxo-4-phenyl-3-pyrrolidinecarboxylic acid 2-[(1 E/Z)-2,3-dihydro-6-methoxy-1H-inden-1-ylidene]hydrazide (46)
2-oxo-4-phenyl-3-pyrrolidinecarboxylic acid 2-[(1 E/Z)-5-bromo-2,3-dihydro-1H-inden-1-ylidene]hydrazide (47)
2-oxo-4-phenyl-3-pyrrolidinecarboxylic acid 2-[(1 E/Z)-2,3-dihydro-6-methyl-1H-inden-1-ylidene]hydrazide (48)
2-oxo-4-phenyl-3-pyrrolidinecarboxylic acid 2-[(1 E/Z)-2,3-dihydro-4,5-dimethoxy-1H-inden-1-ylidene]hydrazide (49)
2-oxo-4-phenyl-3-pyrrolidinecarboxylic acid 2-[(E/Z)-1-(2,4-dimethoxyphenyl)ethylidene]hydrazide (50)
2-oxo-4-phenyl-3-pyrrolidinecarboxylic acid 2-[(E/Z)-1-(2,6-difluorophenyl)ethylidene]hydrazide (51)
2-oxo-4-phenyl-3-pyrrolidinecarboxylic acid 2-[(E/Z)-1-(3-fluoro-4-methoxyphenyl)ethylidene]hydrazide (52)
2-oxo-4-phenyl-3-pyrrolidinecarboxylic acid 2-[(E/Z)-1-(3,4-difluorophenyl)ethylidenelhydrazide (53)
2-oxo-4-phenyl-3-pyrrolidinecarboxylic acid 2-[(4 E/Z)-6-fluoro-2,3-dihydro-4H-1-benzothiopyran-4-ylidene]hydrazide (54)
2-oxo-4-phenyl-3-pyrrolidinecarboxylic acid 2-[(E/Z)-1-(2,3-difluorophenyl)ethylidene]hydrazide (55)
2-oxo-4-phenyl-3-pyrrolidinecarboxylic acid 2-[(4 E/Z)-2,3-dihydro-6-methyl-4H-1-benzopyran-4-ylidene]hydrazide (56)
2-oxo-4-phenyl-3-pyrrolidinecarboxylic acid 2-[(E/Z)-1-(2,3,4,5,6-pentafluorophenyl)ethylidene]hydrazide (57)
2-oxo-4-phenyl-3-pyrrolidinecarboxylic acid 2-[(E/Z)-1-[3-(acetylamino)phenyl]ethylidene]hydrazide (58)
2-oxo-4-phenyl-3-pyrrolidinecarboxylic acid 2-[(E/Z)-1-(1-methyl-1H-pyrrol-3-yl)ethylidene]hydrazide (59)
2-oxo-4-phenyl-3-pyrrolidinecarboxylic acid 2-[(E/Z)-1-(2,4-dimethyl-1H-pyrrol-3-yl)ethylidene]hydrazide (60)
2-oxo-4-phenyl-3-pyrrolidinecarboxylic acid 2-[(E/Z)-1-(1-methyl-1H-pyrrol-2-yl)ethylidene]hydrazide (61)
2-oxo-4-phenyl-3-pyrrolidinecarboxylic acid 2-[(E/Z)-1-(3-thienyl)ethylidene]hydrazide (62)
2-oxo-4-phenyl-3-pyrrolidinecarboxylic acid 2-[(1 E/Z)-3,4-dihydro-7-nitronaphthalenylidene]hydrazide (63)
2-oxo-4-phenyl-3-pyrrolidinecarboxylic acid 2-[(1 E/Z)-6-bromo-2,3-dihydro-1H-inden-1-ylidene]hydrazide (64)
2-oxo-4-phenyl-3-pyrrolidinecarboxylic acid 2-[(1 E/Z)-5-chloro-2,3-dihydro-6-methoxy-1H-inden-1-ylidene]hydrazide (65)

An aspect of this invention is a method for treating or preventing a disease or disorder related to abnormal PAR2 activity comprising administering a therapeutically effective amount of one or more of the compounds listed above.

### DETAILED DESCRIPTION

Definitions

As used herein, any “R” group(s) such as, without limitation, R, R^{a}and R^{b}, is(are) independently selected from the group consisting of hydrogen, alkyl, alkenyl, alkynyl, cycloalkyl, aryl, heteroaryl (bonded to the indicated group at a ring carbon atom) and heteroalicyclyl (likewise bonded to the indicated group at a ring carbon atom), as these groups are defined herein. If two “R” groups are covalently bonded to the same atom or to adjacent atoms, then they may be “taken together” as defined herein to form a cycloalkyl, aryl, heteroaryl or heteroalicyclyl group.

When a group of this invention is described as being “optionally substituted” that group may be unsubstituted or substituted with one or more of the indicated substituents. Likewise, when a group is described as being “unsubstituted or substituted,” if substituted, the substituent may be one or more of the indicated substitutents.

As used herein, “Cₘ to Cₙ,” in which “m” and “n” are integers, refers to the number of carbon atoms in an alkyl, alkenyl or alkynyl group or the number of carbon atoms in the ring of a cycloalkyl or cycloalkenyl group. That is, the alkyl, alkenyl, alkynyl, ring of the cycloalkyl or ring of the cycloalkenyl can contain from “m” to “n”, inclusive, carbon atoms. Thus, for example, a “C₁ to C₄ alkyl” group refers to all alkyl groups having from 1 to 4 carbons, that is, CH₃—, CH₃CH₂—CH₃CH₂CH₂—, CH₃CH(CH₃)—, CH₃CH₂CH₂CH₂—, CH₃CH₂CH(CH₃)— and (CH₃)₃CH—. If no “m” and “n” are designated with regard to an alkyl, alkenyl, alkynyl, cycloalkyl or cycloalkenyl group, the broadest range described in these definitions is to be assumed.

As used herein, “aryl” refers to a carbocyclic (all carbon) ring or two or more fused rings (rings that share two adjacent carbon atoms) that have a fully delocalized pi-electron system. Examples of aryl groups include, but are not limited to, benzene, naphthalene and azulene.

As used herein, “heteroaryl” refers to a ring or two or more fused rings that contain(s) one or more heteroatoms selected from the group consisting of nitrogen, oxygen and sulfur in the ring and that have a fully delocalized pi-electron system. Examples of heteroaryl rings include, but are not limited to, furan, thiophene, phthalazinone, pyrrole, oxazole, thiazole, imidazole, pyrazole, isoxazole, isothiazole, triazole, thiadiazole, pyran, pyridine, pyridazine, pyrimidine, pyrazine and triazine.

As used herein, “alkyl” refers to a straight or branched chain fully saturated (no double or triple bonds) hydrocarbon group. An alkyl group of this invention may comprise from 1 to 20 carbon atoms, that is, m=1 and n=20. An alkyl group herein may also be of medium size having 1 to 10 carbon atoms. It is presently preferred that an alkyl group of this invention be a lower alkyl having 1 to 5 carbon atoms. Examples of alkyl groups include, without limitation, methyl, ethyl, n-propyl, isopropyl, n-butyl, iso-butyl, sec-butyl, tert-butyl, amyl, tert-amyl, hexyl, heptyl, octyl, nonyl, decyl, undecyl and dodecyl.

An alkyl group of this invention may be substituted or unsubstituted. When substituted, the substituent group(s) is(are) one or more independently selected from cycloalkyl, aryl, heteroaryl, heteroalicyciyl, hydroxy, protected hydroxyl, alkoxy, aryloxy, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, trihalomethanesulfonyl, —NR^{a}R^{b} and protected amino.

As used herein, “alkenyl” refers to an alkyl group that contains in the straight or branched hydrocarbon chain one or more double bonds. An alkenyl group of this invention may be unsubstituted or substituted. When substituted, the substituent(s) may be selected from the same groups disclosed above with regard to alkyl group substitution.

As used herein, “alkynyl” refers to an alkyl group that contains in the straight or branched hydrocarbon chain one or more triple bonds. An alkynyl group of this invention may be unsubstituted or substituted. When substituted, the substituent(s) may be selected from the same groups disclosed above with regard to alkyl group substitution.

As used herein, “acyl” refers to an “RC(═O)—” group with R as defined above.

As used herein, “cycloalkyl” refers to a completely saturated (no double bonds) hydrocarbon ring. Cycloalkyl groups of this invention may range from C₃ to C₈. A cycloalkyl group may be unsubstituted or substituted. If substituted, the substituent(s) may be selected from those indicated above with regard to substitution of an alkyl group.

As used herein, “cycloalkenyl” refers to a cycloalkyl group that contains one or more double bonds in the ring although, if there is more than one, they cannot form a fully delocalized pi-electron system in the ring (otherwise the group would be “aryl,” as defined herein). A cycloalkenyl group of this invention may unsubstituted or substituted. When substituted, the substituent(s) may be selected from the same groups disclosed above with regard to alkyl group substitution.

As used herein, “heteroalicyclic” or heteroalicyclyl” refers to a ring or two or more fused rings having in the ring system one or more heteroatoms independently selected from nitrogen, oxygen and sulfur. The rings may also contain one or more double bonds provided that they do not form a fully delocalized pi-electron system in the rings. Heteroalicyclyl groups of this invention may be unsubstituted or substituted. When substituted, the substituent(s) may be one or more groups independently selected from the group consisting of halogen, hydroxy, protected hydroxy, cyano, nitro, alkyl, alkoxy, acyl, acyloxy, carboxy, protected carboxy, amino, protected amino, carboxamide, protected carboxamide, alkylsulfonamido and trifluoromethanesulfonamido.

An “O-carboxy” group refers to a “RC(═O)O—” group with R as defined above.

A “C-carboxy” group refers to a “—C(═O)OH” group.

An “acetyl” group refers to a CH₃C(═O)— group.

A “trihalomethanesulfonyl” group refers to an “X₃CSO₂—” group wherein X is a halogen, i.e., fluorine, chlorine, bromine or iodine.

A “cyano” group refers to a “—CN” group.

An “isocyanato” group refers to an “—NCO” group.

A “thiocyanato” group refers to a “—CNS” group.

An “isothiocyanato” group refers to an “—NCS” group.

A “sulfinyl” group refers to an “—S(═O)—R” group with R as defined above.

A “sulfonyl” group refers to an “SO₂R” group with R as defined above.

An “S-sulfonamido” group refers to a “—SO₂NR^{a}R^{b}” group with R^{a} and R^{b} as defined above.

An “N-sulfonamido” group refers to a “RSO₂N(R^{a})—” group with R and R^{a} as defined above.

A “trihalomethanesulfonamido” group refers to an “X₃CSO₂N(R)—” group with X as halogen and R as defined above.

An “O-carbamyl” group refers to a “—OC(═O)NR^{a}R^{b}” group with R^{a} and R^{b} as defined above.

An “N-carbamyl” group refers to an “ROC(═O)NR^{a}—” group with R^{a} and R as defined above.

An “O-thiocarbamyl” group refers to a “—OC(═S)—NR^{a}R^{b}” group with R^{a} and R^{b} as defined above.

An “N-thiocarbamyl” group refers to an “ROC(═S)NR^{a}—” group with R^{a} and R as defined above.

A “C-amido” group refers to a “—C(═O)NR^{a}R^{b}” group with R^{a} and R^{b} as defined above.

An “N-amido” group refers to a “RC(═O)NR^{a}—” group with R and R^{a} as defined above.

The term “perhaloalkyl” refers to an alkyl group in which all the hydrogen atoms are replaced by halogen atoms.

As used herein, an “ester” refers to a “—C(═O)OR” group with R as defined above.

As used herein, an “amide” refers to a “—C(═O)NR^{a}R^{b}” group with R^{a} and R^{b} as defined above.

Any unsubstituted or monosubstituted amine group of a compound herein can be converted to an amide, any hydroxyl group can be converted to an ester and any carboxyl group can be converted to either an amide or ester using techniques well-known to those skilled in the art (see, for example, Greene and Wuts, Protective Groups in Organic Synthesis, 3^{rd} Ed., John Wiley & Sons, New York, N.Y., 1999). Such amides and esters are within the scope of this invention.

As used herein, the phrase “taken together” when referring to two “R” groups means that the “R” groups are joined together to form a cycloalkyl, aryl, heteroaryl or heteroalicyclyl group. For example, without limitation, if R^{a} and R^{b} of an NR^{a}R^{b} group are indicated to be “taken together,” it means that they are covalently bonded to one another at their terminal atoms to form a ring:

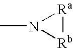

It is understood that, in any compound of this invention having one or more chiral centers, if an absolute stereochemistry is not expressly indicated, then each center may independently be R or S or a mixture thereof. In addition it is understood that, in any compound of this invention having one or more double bond(s) generating geometrical isomers that can be defined as E or Z each double bond may independently be E or Z a mixture thereof.

As used herein, “pharmaceutically acceptable salt” refers to a salt of a compound that does not cause significant irritation to a patient to which it is administered and does not abrogate the biological activity and properties of the compound. Pharmaceutical salts can be obtained by reaction of a compound disclosed herein with an acid or base. Base-formed salts include, without limitation, ammonium salt (NH₄⁺); alkali metal, such as, without limitation, sodium or potassium, salts; alkaline earth, such as, without limitation, calcium or magnesium, salts; salts of organic bases such as, without limitation, dicyclohexylamine, N-methyl-D-glucamine, tris(hydroxymethyl)methylamine; and salts with the amino group of amino acids such as, without limitation, arginine and lysine. Useful acid-based salts include, without limitation, hydrochlorides, hydrobromides, sulfates, nitrates, phosphates, methanesulfonates, ethanesulfonates, p-toluenesulfonates and salicylates.

As used herein, a “prodrug” refers to a compound that may not be pharmaceutically active but that is converted into an active drug in vivo. Prodrugs are often useful because they may be easier to administer than the parent drug. They may, for example, be bioavailable by oral administration whereas the parent drug is not. The prodrug may also have better solubility than the active parent drug in pharmaceutical compositions. An example, without limitation, of a prodrug would be a compound disclosed herein, which is administered as an ester (the “prodrug”) to facilitate absorption through a cell membrane where water solubility is detrimental to mobility but which then is metabolically hydrolyzed to a carboxylic acid (the active entity) once inside the cell where water-solubility is beneficial. A further example of a prodrug might be a short peptide (polyaminoacid) bonded to an acid group where the peptide is metabolized in vivo to reveal the active parent.

As used herein, the term “complement” refers to a oligonucleotide or polynucleotide that hybridizes by base-pairing, adenine to tyrosine and guanine to cytosine, to another oligonucleotide. The hybridized oligonucleotides are then said to be complementary.

As used herein, to “modulate” the activity of PAR2 means either to activate it, i.e., to increase its cellular function over the base level measured in the particular environment in which it is found, or deactivate it, i.e., decrease its cellular function to less than the measured base level in the environment in which it is found and/or render it unable to perform its cellular function at all even in the presence of a natural binding partner. A natural binding partner is an endogenous molecule that is an agonist for the receptor.

As used herein, to “detect” changes in the activity of PAR2 or of a PAR2 sub-type refers to the process of analyzing the result of an experiment using whatever analytical techniques are best suited to the particular situation. In some cases simple visual observation may suffice, in other cases the use of a microscope, visual or UV light analyzer or specific protein assays may be required. The proper selection of analytical tools and techniques to detect changes in the activity of PAR2 or a PAR2 sub-type are well-known to those skilled in the art.

As used herein, an “agonist” refers to a compound that binds to a receptor to from a complex that elicits the full pharmacological response associated with that particular receptor. That is, an agonist for PAR2 may elicit, without limitation, the following responses: mobilization of intracellular calcium, stimulation of phosphatidyl inositol turnover or stimulation of cellular proliferation.

As used herein, “partial agonist” refers to a compound that has an affinity for a receptor but, unlike a full agonist, when bound to the receptor it elicits only a small degree of the pharmacological response normally associated with the receptor even if a large fraction of receptors are occupied by the compound.

As used herein, “inverse agonist” refers to a compound that inhibits the constitutive activity, i.e., activity that exists in the absence of any agonist, of a receptor such that the compound is not technically an antagonist but, rather, is an agonist with negative intrinsic activity.

As used herein, “antagonist” refers to a compound that binds to a receptor to form a complex that does not give rise to any response, as if the receptor were unoccupied. An antagonist may bind reversibly or irreversibly, effectively eliminating the activity of the receptor permanently or at least until the antagonist is metabolized or dissociates or is otherwise removed by a biological process.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes; and, in particular, humans.

As used herein, a “patient” refers to a subject that is being treated by an M.D. or a D.V.M. to attempt to cure, or at least ameliorate the effects of, a particular disease or disorder or to prevent the disease or disorder from occurring in the first place.

As used herein, a “therapeutically effective amount” refers to an amount of a compound that elicits the desired biological or medicinal response in an subject.

As used herein, a “pharmaceutical composition” refers to a mixture of a compound of this invention with other chemical components such as diluents, carriers or other excipients. A pharmaceutical composition may facilitate administration of the compound to a subject. Many techniques of administering a compound are known in the art, such as, without limitation, oral, intramuscular, intra-ocular, intra-nasal, parenteral, intravenous and topical. Pharmaceutical compositions will generally be tailored to the specific intended route of administration.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability, etc., to the composition. A “diluent” is a type of excipient.

Discussion

Synthesis

General synthetic routes to the compounds of this invention are shown in Schemes 1-8. The routes shown are illustrative only and are not intended, nor are they to be construed, to limit the scope of this invention in any manner whatsoever. Those skilled in the art will be able to recognize modifications of the disclosed synthesis and to devise alternate routes based on the disclosures herein; all such modifications and alternate routes are within the scope of this invention. 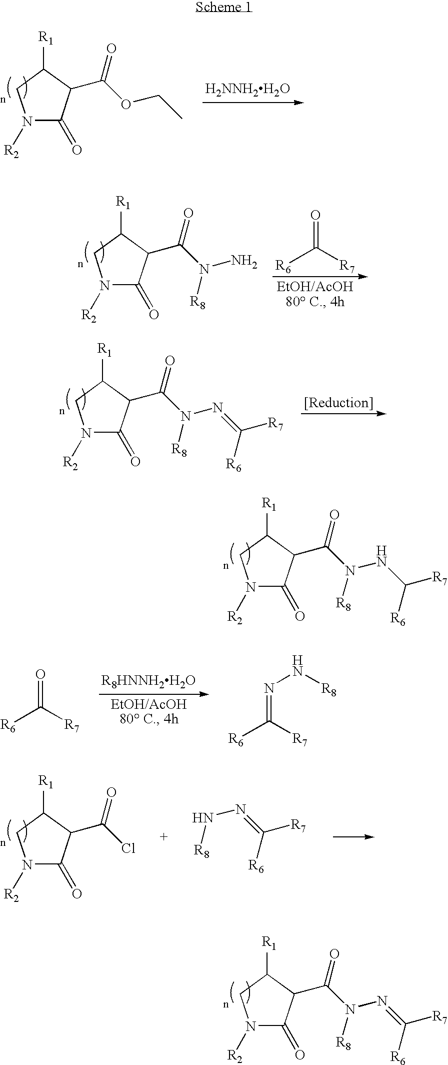 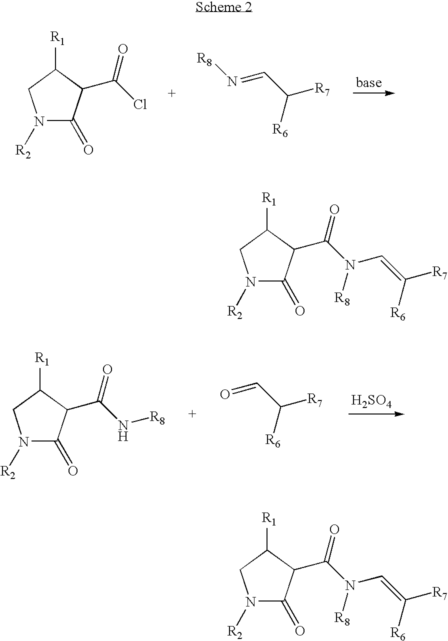 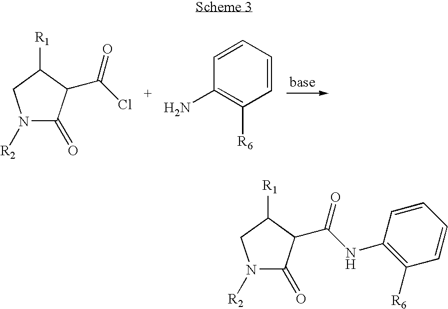 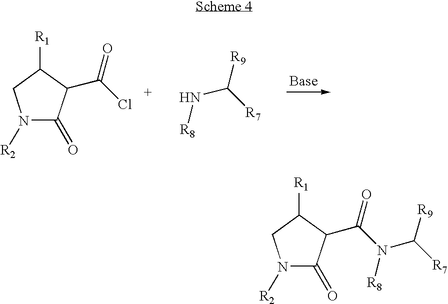 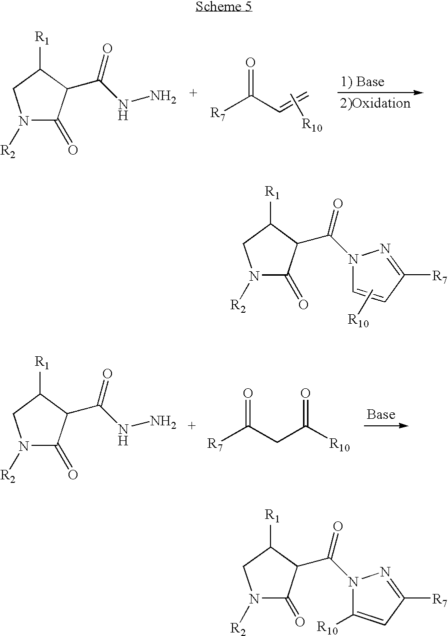 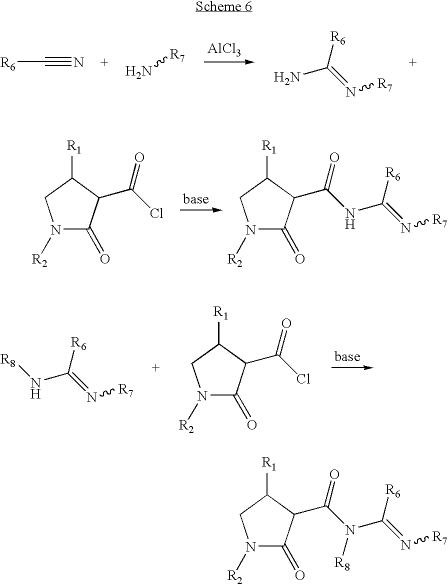 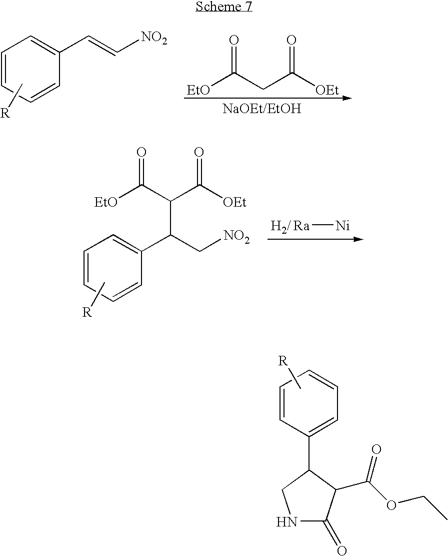 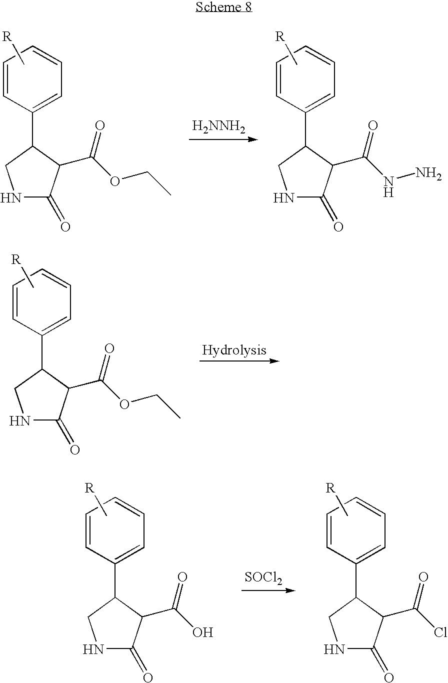
Utility of PAR2 and Compounds Modulating its Activity

Disclosed herein is the use of PAR2 or a PAR2 subtype as a screening tool to identify compounds effective in treating or preventing diseases and disorders including, but not limited to, diseases and disorders of the lung such as asthma, chronic obstructive pulmonary disease, lung cancer and pneumonitis; diseases and disorders of the stomach, small intestine, and large intestine such as gastric ulcers, colitis, inflammatory bowel syndrome, Crohn's disease, gastric and intestinal motilit, colon cancer, cancer of the stomach, and cancer of the intestine; diseases and disorders of the joints such as rheumatoid arthritis; diseases and disorders of the central nervous system such as Alzheimer's disease, encephalitis, meningitis, ischemia and stroke; diseases and disorders of the skin such as dermatitis, psoriasis, pruritis, dermatitis, eczema, seborrhea, wounds, and melanoma; diseases and disorders of the cardiovascular system such as hypertension, atherosclerosis, angina, congestive heart failure, myocarditis and cardiac ischemia; diseases and disorders of the renal (kidney) system such as glomerular kidney disease, kidney cancer and renal failure; diseases and disorders of the hepatic (liver) system such as hepatitis and liver cancer; disease and disorders of the prostatic system such as benign prostatic hyperplasia and prostate cancer; diseases and disorders of the pancreas such as pancreatitis, pancreatic cancer and diabetes; diseases and disorders of the eye such as glaucoma, retinitis pigmentosa, cataracts and macular degeneration; diseases and disorders of the musculoskeletal system such as osteoporosis and Paget's disease; acute and chronic pain, acute and chronic inflammation; dry eye; dry mouth and Sjogren's syndrome. The use of PAR2 or a PAR2 subtype may comprise: a) contacting a recombinant cell with a test compound, where the recombinant cell comprises a recombinant nucleic acid expressing PAR2, provided that the cell does not have functional PAR2 expression from endogenous nucleic acid, and b) determining the ability of the test compound to affect one or more activities of PAR2, and comparing that ability with the ability of the test compound to affect the one or more PAR2 activities in a cell not comprising the recombinant nucleic acid; where the recombinant nucleic acid comprises a PAR2 nucleic acid selected from the group consisting of: i) nucleic acid of SEQ ID NO:1, ii) nucleic acid encoding the amino acid SEQ ID NO:2, iii) a derivative of either nucleic acid molecule in i) or ii), where the derivative encodes a receptor having one or more activities of PAR2 and comprises at least 20 contiguous nucleotides which can hybridize under stringent hybridization conditions to the compliment of the nucleic acid of SEQ ID NO:1.

The PAR2 nucleic acid of this invention encodes the amino acid sequence of a SEQ ID NO:2 derivative comprising at least 20 contiguous nucleotides which can hybridize under stringent conditions to a complement of at least 20 contiguous nucleotides of a polynucleotide that encodes the amino acid sequence of SEQ ID NO:2.

The above derivative can alternatively comprise at least 50, at least 100, at least 150, at least 200, at least 250, at least 300, at least 350, at least 400, at least 450, at least 500, at least 600, at least 700, at least 800, at least 900, at least 1000, at least 1100, at least 1200, at least 1300, at least 1400, at least 1500, at least 1600, at least 1700, at least 1800, at least 1900, at least 2000, at least 2100, at least 2200, at least 2300, at least 2400, or at least 2500, contiguous nucleotides which can hybridize under stringent hybridizations conditions to a complement of contiguous nucleotides encoding the amino acid sequence of SEQ ID NO:2.

The compounds of this invention may be used to treat acute and chronic inflammation by administering to a patient an effective amount of at least one compound of this invention, wherein the compound activates a PAR2 subtype.

Likewise, the compounds of this invention may be used to treat or prevent inflammation by administering to a patient suffering from inflammation an effective amount of at least one compound of this invention, whereby one or more symptoms of the inflammation is reduced.

The compounds of this invention preferably selectively modulate PAR2 or a PAR2 subtype without affecting or minimally affecting other biological pathways. Preferably at present, the modulation comprises activation of the PAR2 or PAR2 subtype, i.e., by being an agonist thereof.

Inflammation may be treated in a patient by administering an therapeutically effective amount of a compound of this invention. An inflammatory response may result, without limitation, from the activation of leukocytes, which comprises leukocyte migration and generation of reactive oxygen species to evoke vascular leakage or edema. The inflammatory response may also result from activation of blood monocytes and neutrophils that infiltrate the affected tissue or organ and in turn activate inflammatory mediators. The inflammatory response may be associated, without limitation, with rheumatoid arthritis, Alzheimer's disease, asthma, chronic obstructive pulmonary disease, gastric ulcers, colitis, inflammatory bowel syndrome, pancreatitis, hepatitis, encephalitis, dermatitis, physical injury or trauma or radiation exposure.

A vasocontractive response may be treated, i.e., reduced or eliminated) or prevented by administering to a patient in need thereof a therapeutically effective amount of a compound of this invention. The vasocontractive response or condition may be related to a renal hemodynamic disease such as, without limitation, glomerular disease or to a cardiovascular disease such as, without limitation, hypertension, congestive heart failure, atherosclerosis, myocarditis, myocardial infarction, or myocardial ischemia.

Likewise, a vasoconstrictive response may be treated, that is, reduced or eliminated, or prevented by administering to a patient in need thereof a therapeutically effective amount of a compound of this invention. The vasoconstrictive response may be associated with a disease or disorder such as, without limitation, asthma, anaphylactic shock, allergic reactions, inflammation, rheumatoid arthritis, gout, psoriasis, allergic rhinitis, adult respiratory distress syndrome, Crohn's disease, endotoxin shock, traumatic shock, hemmorrhagic shock, bowel ischemic shock, renal glomerular disease, benign' prostatic hypertrophy, inflammatory bowel disease, myocardial ischemia, myocardial infarction, circulatory shock, brain injury, systemic lupus erythematosus, chronic renal disease, glomular kidney disease, cardiovascular disease or hypertension.

Acute or chronic pain may be treated or prevented by administering to a patient a therapeutically effective amount of a compound or compounds of this invention.

Diseases and disorders of the eye such as, without limitation, glaucoma, cataracts, macular degeneration and dry eye may be treated by administering to a patient in need thereof a therapeutically effective amount of compound of this invention.

Dry mouth caused by a side effect of a medication or a disease or disorder such as, without limitation, Sjogren's syndrome may also be treated or prevented by administration of a therapeutically effective amount of a compound or compounds of this invention to a patient in need thereof.

Diseases of the bone, such as osteoporosis and Paget's disease, may also be treated or prevented by administration of a therapeutically effective amount of a compound or compounds of this invention to a patient in need thereof.

Acute and chronic pain may also be treated or prevented by administering to a patient in need thereof a therapeutically effective amount of a compound or compounds of this invention, whereby one or more symptoms of the pain are reduced.

It is presently preferred that a compound of this invention be selective for PAR2 or a PAR2 subtype; that is, that it bind only to PAR2 or the subtype such that its therapeutic effect can be directly related to modulation of PAR2 or PAR2 subtype activity and no other.

A method herein of identifying a compound that modulates the activity of PAR2 or a PAR2 subtype may comprise contacting PAR2 with a compound of this invention and detecting any change in the activity level of the PAR2.

Further, a method of identifying a compound which modulates activity of the PAR2 may comprise, under this invention, culturing cells that express PAR2; contacting the cells with a compound of this invention and detecting any change in the activity of PAR2. If desired, the cultured cells may be engineered to over-express PAR2.

The compounds of this invention may be agonists, partial agonists, inverse agonists or antagonists, preferably at present specific agonists, inverse agonists or antagonists of PAR2 or a PAR2 subtype. thus affecting biological processes involving PAR2 and thereby being useful to further elucidate the manner of participation of PAR2 in those biological processes.

Pharmaceutics

The compounds of this invention can be administered to a human patient per se, or in a pharmaceutical composition containing carrier(s), diluent(s) or other excipients. They may also be mixed with other active ingredients as a combination therapy. Techniques for formulation and administration of the compounds may be found in “Remington's Pharmaceutical Sciences,” Mack Publishing Co., Easton, Pa., 18th edition, 1990.

Suitable routes of administration include, without limitation, oral, rectal, transmucosal, intestinal, parenteral, intramuscular, subcutaneous, intravenous, intramedullary, intrathecal, direct intraventricular, intraperitoneal, intranasal, intraocular and as an aerosol inhalant.

It is also possible to administer a compound of this invention locally rather than systemically by, for example, injection directly into the area of pain or inflammation. The compound may be administered as a depot or sustained release formulation. The compound(s) may also be administer in a targeted drug delivery system, for example, in a liposome coated with a tissue-specific antibody that will selectively deliver the liposome to the targeted organ.

The pharmaceutical compositions disclosed herein may be manufactured by procedures well-known in the art, e.g., by means of conventional mixing, dissolving, granulating, dragee-forming, levigating, emulsifying, encapsulating, entrapping or tablet-forming processes.

Pharmaceutical compositions for use in accordance with the present disclosure may be formulated in conventional ways using one or more pharmaceutically acceptable carriers, diluents and other excipients that facilitate processing of the active compounds into preparations. The formulation will depend on the selected route of administration. Any of the well-known techniques and excipients set forth in Remington's as well as novel techniques and excipients as dictated by the particular case may be used.

For injection, the agents disclosed herein may be formulated in aqueous solutions, preferably in physiologically compatible buffers such as Hank's solution, Ringer's solution, or physiological saline buffer.

For transmucosal administration, penetrants appropriate to the barrier to be permeated are used in the formulation. Such penetrants are generally known in the art.

For oral administration, the compounds can be formulated by combining the active compounds with pharmaceutically acceptable carriers well known in the art. Such carriers enable the compounds disclosed herein to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient(s) with one or more compounds herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). Disintegrating agents may be added, such as cross-linked polyvinyl pyrrolidone, agar, or alginic acid or a salt thereof such as sodium alginate.

Dragee cores may be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations, which can be used orally, include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds herein are conveniently delivered in the form of an aerosol spray in pressurized packs or a nebulizer, with a suitable propellant, e.g., dichlorodifluoromethane, trichlorofluoromethane, dichlorotetrafluoroethane, carbon dioxide or other biocompatible gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration either by bolus injection or continuous infusion. Formulations for injection may be presented in unit dosage form, e.g., in ampoules or in multi-dose containers. A preservative may be added. The composition may take such forms as a suspension, solution or emulsion in oily or aqueous vehicles, and may contain formulatory agents such as suspending, stabilizing and/or dispersing agents.

Pharmaceutical formulations for parenteral administration include aqueous solutions of water soluble forms of a compound herein. Alternatively, the compound may be prepared as an oily suspension for injection. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension such as sodium carboxymethyl cellulose, sorbitol, or dextran. Optionally, the suspension may also contain suitable stabilizers or agents, which increase the solubility of the compounds to allow for the preparation of highly concentrated solutions.

A compound herein may be in powder form for constitution with a suitable vehicle, e.g., sterile pyrogen-free water, before use.

A compound herein may also be formulated in rectal compositions such as suppositories or retention enemas containing conventional suppository bases such as cocoa butter or other glycerides.

The compounds may also be formulated as depot preparations. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

A pharmaceutical carrier for the compounds disclosed herein may comprise a co-solvent system such as, without limitation, a mixture comprising benzyl alcohol, a nonpolar surfactant, a water-miscible organic polymer, and an aqueous phase. A common co-solvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. The proportions of the solvents in the co-solvent system may be varied considerably without deleteriously affecting its solubility and toxicity characteristics. Furthermore, the components of the co-solvent system may be varied: For example, other low-toxicity non-polar surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may be used.

Other delivery systems for pharmaceutical compounds may be employed. Liposomes and emulsions are well-known examples of delivery vehicles for hydrophobic drugs. Certain organic solvents such as dimethylsulfoxide may be employed as carriers, although usually at the cost of greater toxicity. Additionally, the compounds may be delivered using a sustained-release system, such as semi-permeable matrices of solid hydrophobic polymers containing the therapeutic agent. Various sustained-release materials and techniques have been established and are well known to those skilled in the art. Sustained-release capsules may, depending on their chemical nature, release the compounds over a couple of hours up to many months.

Many of the compounds herein may be provided as salts with pharmaceutically acceptable counterions. Pharmaceutically compatible salts may be formed with many acids including, but not limited to, hydrochloric, sulfuric, acetic, lactic, tartaric, malic, and succinic acid. Salts tend to be more soluble in aqueous solution than the corresponding free acids or base forms of the compounds herein.

Pharmaceutical compositions suitable for use in the methods disclosed herein include compositions where the compound herein is contained in an amount effective to achieve its intended purpose. Thus, a therapeutically effective amount means an amount of compound effective to prevent, alleviate, ameliorate or cure symptoms of a disease or disorder or to prolong the survival of the patient being treated. Determination of a therapeutically effective amount is well within the capability of those skilled in the art, especially in light of the detailed disclosure provided herein.

The exact formulation, route of administration and dosage for the pharmaceutical compositions disclosed herein will be selected by the treating physician who is most familiar with the patient's condition. (See e.g., Fingl et al. 1975, in “The Pharmacological Basis of Therapeutics”, Ch. 1 p. 1). Typically, the dose range of the composition administered to the patient will be from about 0.5 to about 1000 mg/kg, preferably at present from about 1 to about 500 mg/kg, still more preferably at present about 10 to 500 mg/kg or even more preferably at present from about 50 to about 100 mg/kg of body weight. The dosage may be unitary or a series of two or more doses administered over time. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

Although the exact dosage will be determined on a compound-by-compound basis, some generalizations regarding can be made. The daily dosage regimen for an adult human patient may be, for example, an oral dose of between about 0.1 mg and about 500 mg of each ingredient, preferably between about 1 mg and about 250 mg or an intravenous, subcutaneous, or intramuscular dose between about 0.01 mg and about 100 mg, preferably between about 0.1 mg and about 60 mg calculated as the free base, the composition being administered 1 to 4 times per day. Alternatively the compositions disclosed herein may be administered by continuous intravenous infusion, preferably at a dose of each ingredient up to 400 mg per day. Thus, the total daily dosage by oral administration of each ingredient will typically be in the range 1 to 2000 mg and the total daily dosage by parenteral administration will typically be in the range 0.1 to 400 mg. The compounds may be administered as a continuous course of therapy, for example for a week, a month or, in some cases, for a period of years.

Dosage amount and interval may be adjusted individually to provide plasma levels of the active compound taht are sufficient to maintain a minimum effective concentration (MEC). The MEC will vary for each compound but can be estimated from in vitro data. Dosages necessary to achieve the MEC will depend on the individual patient and the route of administration. HPLC assays or bioassays can be used to determine plasma concentrations.

Dosage intervals can also be determined using MEC value. Compositions should be administered using a regimen that maintains plasma levels above the MEC for 10-90%, preferably at present between 30-90% and most preferably at present between 50-90% of the time.

In cases of local administration or selective uptake, the effective local concentration of the drug may not be related to plasma concentration.

The amount of a compound herein administered will depend on the patient being treated, his/her weight and particular biochemistry, the severity of the affliction, the manner of administration and the judgment of the prescribing physician.

Compositions containing the compounds herein may be presented in a pack or dispenser device, which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound disclosed herein formulated in a compatible pharmaceutical carrier may also be prepared, placed in an appropriate container, and labeled for treatment of an indicated condition.

### EXAMPLES

The examples that follow are provided by way of illustration only and are not intended, nor are they to be construed, as limiting the scope of this invention in any manner whatsoever.

Analytical procedure

Analyses were performed on a combined prep/analytical Waters/Micromass system consisting of a ZMD single quadropole mass spectrometer equipped with electro-spray ionization interface. The HPLC system consisted of a Waters 600 gradient pump with on-line degassing, a 2700 sample manager and a 996 PDA detector.

Separation was performed on an X-Terra MS C18, 5 μm 4.6×50 mm column using two buffers. Buffer A was 10 mM ammonium acetate in water and buffer B was 10 mM ammonium acetate in acetonitrile/water 95/5. A gradient was run from 10% B to 100% B over 10 min, the eluent held at 100% B for 1 min and then re-equilibrated for 6 min. The system was operated at 1 ml/min.

For some analyses, the gradient used was from 30% B to 100% B over 7 min with a hold time at 100% B of 1 min followed by re-equilibration for 5.5 min. Again, the system was operated at 1 ml/min.

### Example 1

### Ethyl 2-oxo-4-phenyl-3-pyrrolidinecarboxylate (1)

2-Oxo-4-phenyl-3-pyrrolidinecarboxylic acid (600 mg, 2.92 mmol) was dissolved in EtOH (5 mL), conc. H₂SO₄ (0.2 mL) was added and the mixture was subjected to microwave radiation (120° C., 600 s). EtOAc was subsequently added and washed with sat. NaHCO₃ and brine, dried (Na₂SO₄) and concentrated. Yield: 498.2 mg (2.14 mmol).

¹H-NMR (400 MHz, CDCl₃) d 7.40-7.20 (m, 5H), 4.23 (q, 2H, J=7.04 Hz), 4.10 (q, 1H, J=8.5 Hz), 3.81 (t, 1H, J=9.1 Hz), 3.55 (d, 1H, J=9.58 Hz), 3.43 (dd, 1H, J=9.58 Hz, 8.12 Hz), 1.27 (t, 3H, J=7.04 Hz).

¹³C-NMR (100 MHz, CDCl₃) d 173.1, 169.5, 140.1, 129.2, 127.8, 127.2, 62.0, 55.6, 48.0, 44.6, 14.4.

### Example 2

### 2-Oxo-4-phenyl-3-pyrrolidinecarboxylic acid hydrazide (2)

Ethyl 2-oxo-4-phenyl-3-pyrrolidinecarboxylate (498 mg, 2.14 mmol) was dissolved in EtOH (5 mL) and hydrazine hydrate (0.5 mL) was added. The mixture was subjected to microwave radiation (120 ° C., 1200 s), concentrated and the resulting white solid was recrystallized from EtOAc with hot filtration Yield: 261.3 mg (1.192 mmol).

¹H-NMR (400 MHz, CD₃OD) d 7.35-7.24 (m, 5H), 4.06 (q, 1H, J=8.8 Hz), 3.76 (m, 1H), 3.43 (m, 2H).

### Example 3

### 2-Oxo-4-phenyl-3-pyrrolidinecarboxylic acid [1-(3-bromo-phenyl)-(E/Z)-ethylidene]-hydrazide (3)

2-Oxo-4-phenyl-3-pyrrolidinecarboxylic acid hydrazide (54.0 mg, 0.246 mmol) and 3-bromoacetophenone (98.1 mg, 0.493 mmol) was taken up in EtOH (2 mL) and AcOH (0.2 mL) and subjected to microwave radiation (120° C., 600 s). The solvent was removed in vacuo and the residue purified by column chromatography (silica, 0-3% MeOH in DCM). Yield: 70.5 mg (0.176 mmol).

¹H-NMR (400 MHz, dmso-d6) (compound is a mixture of isomers) d 10.8, 10.7 (2×s, 1H), 8.10, 8.00, 7.93, 7.80 (4×s, 2H), 7.73 (d, J=8.6 Hz, 1069 1/2H), 7.65 (d, J=8.0 Hz, 1/2H), 7.56 (dd, J=8.0 Hz, 1.0 Hz, 1H), 7.32 (m, 5H), 4.63, 3.87 (2×d, J=10.5 Hz, 1H), 4.01 (m, 1H), 3.67 (m, 1H), 3.32 (m, 1H), 2.23, 2.18 (2×s, 3H).

LC-MS (AP2): purity (UV/MS): 100/100, Rₜ 3.08/3.32 min (two isomers).

### Example 4

### 2-Oxo-4-phenyl-3-pyrrolidinecarboxylic acid [1-(5-bromo-thiophen-2-yl)-(E/Z)-ethylidene]-hydrazide (4)

2-Oxo-4-phenyl-3-pyrrolidinecarboxylic acid hydrazide (21.9 mg, 0.10 mmol) and 2-acetyl-5-bromothiophene (41.0 mg, 0.20 mmol) was taken up in EtOH:AcOH (9:1, 1 mL) and refluxed for 10 h. The solvent was evaporated in vacuo and the residue purified by column chromatography (silica, 0-3% MeOH in DCM). Yield: 26.0 mg.

¹H-NMR (400 MHz, CD₃OD) d 7.35-6.98 (m, 7H), 4.18-4.09 (m, 1H), 3.85-3.77 (m, 2H), 3.45 (q, 1H, J=9.6 Hz), 2.23, 2.17 (2×s, 3H).

LC-MS (AP2): purity (UV/MS): 98/90, Rₜ 3.01/3.23 min (two isomers)

### Example 5

### 2-Oxo-4-phenyl-3-pyrrolidinecarboxylic acid [1-(thiophen-2-yl)-(E/Z)-ethylidene]-hydrazide (5)

2-Oxo-4-phenyl-3-pyrrolidinecarboxylic acid hydrazide (21.9 mg, 0.10 mmol) and 2-acetyl thiophene (25.4 mg, 0.20 mmol) was taken up in EtOH:AcOH (9:1, 1 mL) and refluxed for 10 h. The solvent was evaporated in vacuo and the residue purified by column chromatography (silica, 0-3% MeOH in DCM). Yield: 20.4 mg.

¹H-NMR (400 MHz, CD₃OD) d 7.46-7.00 (m, 8H), 4.16 (m, 1H), 3.82 (m, 2H), 3.44 (m, 1H), 2.30, 2.24 (2×s, 3H)

LC-MS (AP2): purity (UV/MS): 100/100, Rₜ 2.44 min

### Example 6

### 2-Oxo-4-phenyl-3-pyrrolidinecarboxylic acid [1-(4-bromo-thiophen-2-yl)-(E/Z)-ethylidene]-hydrazide (6)

2-Oxo-4-phenyl-3-pyrrolidinecarboxylic acid hydrazide (21.9 mg, 0.10 mmol) and 2-acetyl-4-bromo-thiophene (41.0 mg, 0.20 mmol) was taken up in EtOH:AcOH (9:1, 1 mL) and refluxed for 10 h. The solvent was evaporated in vacuo and the residue purified by column chromatography (silica, 0-3% MeOH in DCM). Yield: 19.3 mg.

LC-MS (AP2): purity (UV/MS): 93/71, Rₜ 3.21 min

### Example 7

### 2-Oxo-4-phenyl-3-pyrrolidinecarboxylic acid [-1-(5-bromo-pyridine-3-yl)-(E/Z)-ethylidene]-hydrazide (7)

2-Oxo-4-phenyl-3-pyrrolidinecarboxylic acid hydrazide (21.9 mg, 0.10 mmol) and 5-bromo-3-acetyl pyridine (40.0 mg, 0.20 mmol) was taken up in EtOH:AcOH (9:1, 1 mL) and refluxed for 10 h. The solvent was evaporated in vacuo and the residue purified by column chromatography (silica, 0-3% MeOH in DCM). Yield: 26.6 mg.

LC-MS (AP2): purity (UV/MS): 99/85, Rₜ 1.95/2.27 min (two isomers)

### Example 8

### 2-Oxo-4-phenyl-3-pyrrolidinecarboxylic acid [1-(4-bromo-phenyl)-(E/Z)-ethylidene]-hydrazide (8)

2-Oxo-4-phenyl-3-pyrrolidinecarboxylic acid hydrazide (21.9 mg, 0.10 mmol) and 4-bromo acetophenone (39.8 mg, 0.20 mmol) was taken up in EtOH:AcOH (9:1, 1 mL) and refluxed for 10 h. Upon cooling the product crystallized and was filtered and washed with cold ethanol. Yield: 26.0 mg.

LC-MS (AP2): purity (UV/MS): 98/100, Rₜ 3.13/3.39 min (two isomers)

### Example 9

### 2-Oxo-4-phenyl-3-pyrrolidinecarboxylic acid [1-(2-bromo-phenyl)-(E/Z)-ethylidene]-hydrazide (9)

2-Oxo-4-phenyl-3-pyrrolidinecarboxylic acid hydrazide (21.9 mg, 0.10 mmol) and 2-bromo acetophenone (39.8 mg, 0.20 mmol) was taken up in EtOH:AcOH (9:1, 1 mL) and refluxed for 10 h. The solvent was evaporated in vacuo and the residue purified by column chromatography (silica, 0-3% MeOH in DCM). Yield: 28.3 mg.

LC-MS (AP2): purity (UV/MS): 99/89, Rₜ 2.77/3.05 min (two isomers)

### Example 10

### 2-Oxo-4-phenyl-3-pyrrolidinecarboxylic acid [1-(3-methoxy-phenyl)-(E/Z)-ethylidene]-hydrazide (10)

2-Oxo-4-phenyl-3-pyrrolidinecarboxylic acid hydrazide (21.9 mg, 0.10 mmol) and 3-methoxy acetophenone (30.0 mg, 0.20 mmol) was taken up in EtOH:AcOH (9:1, 1 mL) and refluxed for 10 h. The solvent was evaporated in vacuo and the residue purified by column chromatography (silica, 0-3% MeOH in DCM). Yield: 27.0 mg.

LC-MS (AP2): purity (UV/MS): 100/73, Rₜ 2.47/2.75 min (two isomers)

### Example 11

### 2-Oxo-4-phenyl-3-pyrrolidinecarboxylic acid [1-(toluen-3-yl)-(E/Z)-ethylidene]-hydrazide (11)

2-Oxo-4-phenyl-3-pyrrolidinecarboxylic acid hydrazide (21.9 mg, 0.10 mmol) and 3-methyl acetophenone (26.8 mg, 0.20 mmol) was taken up in EtOH:AcOH (9:1, 1 mL) and refluxed for 10 h. The solvent was evaporated in vacuo and the residue purified by column chromatography (silica, 0-3% MeOH in DCM). Yield: 19.8 mg.

LC-MS (AP2): purity (UV/MS): 100/100, Rₜ 2.72/3.07 min (two isomers)

### Example 12

### 2-Oxo-4-phenyl-3-pyrrolidinecarboxylic acid [1-(3-trifluoromethyl-phenyl)-(E/Z)-ethylidene]-hydrazide (12)

2-Oxo-4-phenyl-3-pyrrolidinecarboxylic acid hydrazide (21.9 mg, 0.10 mmol) and 3-trifluoromethyl acetophenone (37.6 mg, 0.20 mmol) was taken up in EtOH:AcOH (9:1, 1 mL) and refluxed for 10 h. Upon cooling the product crystallized and was filtered and washed with cold ethanol. Yield: 21.5 mg.

LC-MS (AP2): purity (UV/MS): 100/100, Rₜ 3.25/3.49 min (two isomers)

### Example 13

### 2-Oxo-4-phenyl-3-pyrrolidinecarboxylic acid N′-[1-(3-bromo-phenyl)-ethyl]-hydrazide (13)

2-Oxo-4-phenyl-3-pyrrolidinecarboxylic acid [1-(3-bromo-phenyl)-(E/Z)-ethylidene]-hydrazide (10.0 mg, 0.025 mmol) was dissolved in EtOH (1 mL) and a few drops of AcOH was added followed by NaCNBH₃ (15.7 mg, 0.250 mmol). The mixture was stirred for 2 h at room temperature, EtOAc was added and the solution washed with sat. NaHCO₃, dried over Na₂SO₄, concentrated and the residue run through a plug of silica (5% MeOH/DCM) to give the product. Yield: 9.4 mg, 0.0234 mmol.

¹H-NMR (400 MHz, CDCl₃) d 7.55-7.12 (m, 9H), 4.80-3.91 (m, 2H), 3.77-3.71 (m, 1H), 3.41-3.30 (m, 2H), 1.30, 1.28, 1.27, 1.26 (4×s, 3H).

LC-MS (AP2): purity (UV/MS): 100/95, Rₜ 3.07 min

### Example 14

### Receptor Selection and Amplification Technology Assay

The functional receptor assay, Receptor Selection and Amplification Technology (R-SAT), was used to investigate the pharmacological properties of known and novel PAR2 compounds. R-SAT is disclosed in U.S. Pat. Nos. 5,707,798, 5,912,132, and 5,955,281, all of which are hereby incorporated herein by reference in their entirety, including any drawings.

Briefly, NIH3T3 cells were grown in 96 well tissue culture plates to 70-80% confluence. Cells were transfected for 16-20 h with plasmid DNAs using Polyfect (Qiagen Inc.) using the manufacturer's protocols. R-SATs were generally performed with 4 ng/well of receptor and 20 ng/well of β-galactosidase plasmid DNA. All receptor constructs used were in the pSI-derived mammalian expression vector (Promega Inc). The PAR2 gene was amplified by PCR from genomic DNA using oligodeoxynucleotide primers based on the published sequence (GenBank Accession # Z49993 and Z49994). For large-scale transfections, cells were transfected for 16-20 h, then trypsinized and frozen in DMSO. Frozen cells were later thawed, plated at ˜10,000 cells per well of a 96 half-area well plate that contained drug. With both methods, cells were then grown in a humidified atmosphere with 5% ambient CO₂ for five days. Media was then removed from the plates and marker gene activity was measured by the addition of the β-galactosidase substrate o-nitrophenyl β-D-galactopyranoside (ONPG) in PBS with 0.5% NP-40. The resulting colorimetric reaction was measured using a spectrophotometric plate reader (Titertek Inc.) at 420 nm. All data was analyzed using the XLFit (IDBSm) computer program. Efficacy is the percent maximum activation compared to activation by a control compound (SLIGRLm). pEC₅₀ is the negative of the log(EC₅₀), where EC₅₀ is the calculated as a molar concentration (M) that produces 50% maximal activation.

These experiments provide a molecular profile, or fingerprint, for each of these agents at the human PAR2. As can be seen in Table 1, the compounds tested activated PAR2.

TABLE 1

| Compound | pEC₅₀ | % Efficacy |
|---|---|---|
| 3 | 7.4 | 134 |
| 4 | 6.2 | 87 |
| 5 | 5.8 | 52 |
| 6 | 6.4 | 109 |
| 7 | 6.1 | 106 |

Efficacy is relative to the ligand SLIGRL.

Using the following methods for evaluate the ability of the compounds disclosed herein to bind to the PAR2 receptors can be readily determined in the following assays: Receptor Binding Assay, Determination of Changes in Cytosolic Calcium in Transfected CHO-K1 Cells and Determination of Changes in Inositol Phosphates in Transfected TsA Cells.

### Example 15

### PAR2 Binding Assay

Using the following materials and methods, the ability of the compounds disclosed herein to bind to PAR2 can be readily determined in a receptor binding assay.

1. Grow PAR2-transfected COS cells (or another transfected cell line that does not endogenously express PAR2) in a suitable growth medium in 24-well culture plates.

2. Prepare radiolabeled assay solutions by mixing 245 μl of 0.25 nM [¹²⁵l] SLIGRL working solution with 5 μl of the following: 50 μM unlabeled SLIGRL working solution, 0.25 nM [¹²⁵l] SLIGRL working solution, HEPES buffer only and 50× test compound.

3. Aspirate medium from 24-well plates using a Pasteur pipette attached to a vacuum source. Do not wash cells.

4. Add 250 μl of the radiolabeled assay solution from step 2 to each assay well and incubate plates for 60 min at room temperature (˜22° C.) on an orbital shaker at low speed.

5. Terminate the incubation by aspirating the radioactive solution with a 24-well Brandel cell harvester. Wash the wells three times with 0.5 ml ice-cold HEPES buffer using the cell harvester.

6. Aspirate the solution from the wells with a micropipette and transfer to 12×75-mm polystyrene test tubes. Analyze with a gamma counter (Packard, Cobra II).

7. Determine specific binding and calculate the IC₅₀ values.

### Example 16

### Determination of Changes in Cytosolic Calcium in Transfected CHO-K1 Cells

1. Wash CHO-K1 cells transfected with PAR2 or a control receptor, either at a density of 1-3×10⁶ cells/ml with phosphate-buffered saline.

2. Load cells with 2 μM Fura-2 and analyze with respect to the rise in intracellular calcium in the presence or absence of varying concentration of test compound.

3. The response is compared to that elicited by the application of the standard reference ligand SLIGRL at 100 nM.

Intracellular free calcium concentrations are calculated using the formula:

$${[ {Ca}^{2 +} ]}_{i} = \frac{{K}_{d} ( F - {F}_{min} )}{{F}_{max} - F}$$

where K_{d} for Fura-2 is 224 nM, Fₘₐₓ is the fluorescence in the presence of 0.04% Triton-X100 and Fₘᵢₙ is the fluorescence obtained after the addition of 5 mM EGTA in 30 mM Tris-HCl, pH 7.4.

As shown in Table 2 and FIG. 3, the compounds tested act at human PAR2 to stimulate intracellular calcium mobilization.

### Example 17

### Determination of Changes in Inositol Phosphates in Transfected TsA Cells

Seed tsA cells (a transformed HEK293 cell line) at 10,000 cells/0.1 ml per well of 96-well plates and grow overnight at 37° C. in a humidified 5% CO₂ incubator in DMEM supplemented with 10% fetal calf serum, penicillin (100 units/ml) and streptomycin (100 mg/ml).

Transfect the cells with plasmid DNAs coding receptors and G-protein helpers when needed using PolyFect according to the R-SAT protocol described in the above-referenced U.S. patents. At 18-20 h post-transfection, remove the medium and label the cells overnight with 2 uCi/ml myo-[2-3H]-inositol (0.1 ml/well) freshly made in the culture medium.

Remove the medium and wash cells with Hank's Balanced Salt Solutions (HBSS) containing 1 mM CaCl₂, 1 mM MgCl₂, 20 mM LiCl and 0.1% BSA. The cells are then incubated with ligands for 45 min at 37° C. (0.1 ml/well) and the reaction is stopped by exchanging the buffer with 150 ul/well ice-cold 20 mM formic acid. Add 50 ul/well 0.2M ammonium and store plates at −80° C. or process samples immediately.

Separate total [3 H] inositol phosphates (IPs) by ion-exchange chromatography. The column is loaded with 200 ul of AG 1-X8 resin suspension (50% resin and 50% water) and the cell extracts are applied to the columns. Elute the columns with 1 ml 40 mM ammonium hydroxide (pH9) and elute [3 H] IPs into the 2 ml deep-well blocks with 0.4 ml 2 M ammonium format/0.1 M formic acid. Wash the columns with 0.6 ml water. Transfer the eluants into 7 ml scintillation vials and add 5 ml liquid scintillation cocktail. Mix well, leave the vials in the dark for at least 4 h and count on LS 6500 Multi-purpose Scintillation Counter (3 min/vial). This procedure collects IP1, IP2 and IP3.

### Example 18

### Sequences for PAR2

The DNA sequence encoding PAR2 (SEQ ID NO:1 ) and the polypeptide sequence of PAR2 (SEQ ID NO:2) are:

SEQ ID NO:1
ctcgagcggccgccagtgtgatggatatctgcagaattcgccctttgcgtccagtggagctctgagtttcgaatcgg
tggcggcggattccccgcgcgcccggcgtcggggcttccaggaggatgcggagccccagcgcagcgtggctg
ctgggggccgccatcctgctagcagcctctctctcctgcagtggcaccatccaaggaaccaatagatcctctaaa
ggaagaagccttattggtaaggttgatggcacatcccacgtcactggaaaaggagttacagttgaaacagtctttt
ctgtggatgagttttctgcatctgtcctcactggaaaactgaccactgtcttccttccaattgtctacacaattgtgtttgt
ggtgggtttgccaagtaacggcatggccctgtgggtctttcttttccgaactaagaagaagcaccctgctgtgattta
catggccaatctggccttggctgacctcctctctgtcatctggttccccttgaagattgcctatcacatacatggcaac
aactggatttatggggaagctctttgtaatgtgcttattggctttttctatggcaacatgtactgttccattctcttcatgac
ctgcctcagtgtgcagaggtattgggtcatcgtgaaccccatggggcactccaggaagaaggcaaacattgcc
attggcatctccctggcaatatggctgctgattctgctggtcaccatccctttgtatgtcgtgaagcagaccatcttcat
tcctgccctgaacatcacgacctgtcatgatgttttgcctgagcagctcttggtgggagacatgttcaattacttcctct
ctctggccattggggtctttctgttcccagccttcctcacagcctctgcctatgtgctgatgatcagaatgctgcgatctt
ctgccatggatgaaaactcagagaagaaaaggaagagggccatcaaactcattgtcactgtcctggccatgta
cctgatctgcttcactcctagtaaccttctgcttgtggtgcattattttctgattaagagccagggccagagccatgtct
atgccctgtacattgtagccctctgcctctctacccttaacagctgcatcgacccctttgtctattactttgtttcacatga
tttcagggatcatgcaaagaacgctctcctttgccgaagtgtccgcactgtaaagcagatgcaagtatccctcacc
tcaaagaaacactccaggaaatccagctcttactcttcaagttcaaccactgttaagacctcctattgagttttccag
gtcctcagatgggaattgcacagtaggatgtggaacctgtttaatgttatgaggacgtgtctgaagggcgaattcc
agcacactggcggccgtt

SEQ ID NO:2
MRSPSAAWLLGAAILLAASLSCSGTIQGTNRSSKGRSLIGKVDGTSHVTG
KGVTVETVFSVDEFSASVLTGKLTTVFLPIVYTIVFVVGLPSNGMALWVF
LFRTKKKHPAVIYMANLALADLLSVIWFPLKIAYHIHGNNWIYGEALCNV
LIGFFYGNMYCSILFMTCLSVQRYWVIVNPMGHSRKKANIAIGISLAIWL
LILLVTIPLYVVKQTIFIPALNITTCHDVLPEQLLVGDMFNYFLSLAIGV
FLFPAFLTASAYVLMIRMLRSSAMDENSEKKRKRAIKLIVTVLAMYLICF
TPSNLLLVVHYFLIKSQGQSHVYALYIVALCLSTLNSCIDPFVYYFVSHD
FRDHAKNALLCRSVRTVKQMQVSLTSKKHSRKSSSYSSSSTTVKTSY*

References Cited
1. Hollenberg M D, Compton S J. International Union of Pharmacology. XXVIII. Proteinase-activated receptors. Pharmacol Rev. 2002 June;54(2):203-17
2. Macfarlane S R, Seatter M J, Kanke T, Hunter G D, Plevin R. Proteinase-activated receptors. Pharmacol Rev. 2001 June;53(2):245-82.
3. Nakanishi-Matsui M, Zheng Y W, Sulciner D J, Weiss E J, Ludeman M J, Coughlin S R. PAR3 is a cofactor for PAR4 activation by thrombin. Nature 2000 404:609-13
4. Cocks T M, Fong B, Chow J M, Anderson G P, Frauman A G, Goldie R G, Henry P J, Carr M J, Hamilton J R, Moffatt J D. A protective role for protease-activated receptors in the airways. Nature. 1999 March 11;398(6723):156-60.
5. Fiorucci S, Mencarelli A, Palazzetti B, Distrutti E, Vergnolle N, Hollenberg M D, Wallace J L, Morelli A, Cirino G. Proteinase-activated receptor 2 is an anti-inflammatory signal for colonic lamina propria lymphocytes in a mouse model of colitis. Proc Natl Acad Sci USA. 2001 November 20; 98(24):13936-41
6. Kawabata A, Kinoshita M, Nishikawa H, Kuroda R, Nishida M, Araki H, Arizono N, Oda Y, Kakehi K. The protease-activated receptor-2 agonist induces gastric mucus secretion and mucosal cytoprotection. J Clin Invest. 2001 June; 107(11): 1443-50
7. Cocks T M, Moffatt J D. Protease-activated receptors: sentries for inflammation? Trends Pharmacol Sci. 2000 March; 21(3):103-8
8. Chow J M, Moffatt J D, Cocks T M. Effect of protease-activated receptor (PAR)-1, -2 and -4-activating peptides, thrombin and trypsin in rat isolated airways Br J Pharmacol. 2000 December; 131(8): 1584-91
9. Cocks T M, Moffatt J D. Protease-activated receptor-2 (PAR2) in the airways. Pulm Pharmacol Ther. 2001;14(3): 183-91.
10. Vergnolle N, Wallace J L, Bunnett N W, Hollenberg M D. Protease-activated receptors in inflammation, neuronal signaling and pain. Trends Pharmacol.Sci 2001 March;22(3):146-52
11. Steinhoff M, Vergnolle N, Young S H, Tognetto M, Amadesi S, Ennes H S, Trevisani M, Hollenberg M D, Wallace J L, Caughey G H, Mitchell S E, Williams L M, Geppetti P, Mayer E A, Bunnett N W. Agonists of proteinase-activated receptor 2 induce inflammation by a neurogenic mechanism. Nat Med. 2000 February;6(2):151-8.
12. Fiorucci S, Distrutti E. Role of PAR2 in pain and inflammation. Trends Pharmacol Sci. 2002 April;23(4):153-5
13. Vergnolle N, Bunnett N W, Sharkey K A, Brussee V, Compton S J, Grady E F, Cirino G, Gerard N, Basbaum A l, Andrade-Gordon P, Hollenberg M D, Wallace J L. Proteinase-activated receptor-2 and hyperalgesia: A novel pain pathway. Nat Med. 2001 July;7(7):821-6
14. Mantyh P W, Yaksh T L. Sensory neurons are PARtial to pain. Nat Med. 2001 July;7(7):772-3.
15. Nishikawa H, Kawai K, Tanaka M, Ohtani H, Tanaka S, Kitagawa C, Nishida M, Abe T, Araki H, Kawabata A. Protease-activated receptor-2 (PAR-2)-related peptides induce tear secretion in rats: involvement of PAR-2 and non-PAR-2 mechanisms. J Pharmacol Exp Ther. 2005 January;312(1):324-31.
16. Smith R, Ransjo M, Tatarczuch L, Song S J, Pagel C, Morrison J R, Pike R N, Mackie E J. Activation of protease-activated receptor-2 leads to inhibition of osteoclast differentiation. J Bone Miner Res. 2004 March;19(3):507-16.
17. Jin G, Hayashi T, Kawagoe J, Takizawa T, Nagata T, Nagano I, Syoji M, Abe K. Deficiency of PAR-2 gene increases acute focal ischemic brain injury. J Cereb Blood Flow Metab. 2005 Jan. 12; [Epub ahead of print]
18. Napoli C, De Nigris F, Cicala C, Wallace J L, Caliendo G, Condorelli M, Santagada V, Cirino G. Protease-activated receptor-2 activation improves efficiency of experimental ischemic preconditioning. Am J Physiol Heart Circ Physiol. 2002 June;282(6):H2004-10.
19. Napoli C, Cicala C, Wallace J L, de Nigris F, Santagada V, Caliendo G, Franconi F, Ignarro L J, Cirino G. Protease-activated receptor-2 modulates myocardial ischemia-reperfusion injury in the rat heart. Proc Natl Acad Sci USA. 2000 Mar. 28;97(7):3678-83.
20. Steinhoff M, Neisius U, Ikoma A, Fartasch M, Heyer G, Skov P S, Luger T A, Schmelz M. Proteinase-activated receptor-2 mediates itch: a novel pathway for pruritus in human skin. J Neurosci. 2003 July 16;23(15):6176-80.
21. Sharma A, Tao X, Gopal A, Ligon B, Andrade-Gordon P, Steer M L, Perides G. Protection against acute pancreatitis by activation of protease-activated receptor-2. Am J Physiol Gastrointest Liver Physiol. 2005 Febuary;288(2):G388-95.
22. McGuire J J, Hollenberg M D, Andrade-Gordon P, Triggle C R. Multiple mechanisms of vascular smooth muscle relaxation by the activation of proteinase-activated receptor 2 in mouse mesenteric arterioles. Br J Pharmacol. 2002 January;135(1):155-69.

What is claimed is:

1. A compound having the chemical structure 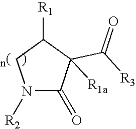
or a pharmaceutically acceptable salt or prodrug thereof, wherein:
n is 1, 2, or 3;
R₁ is selected from the group consisting of hydrogen, alkyl, alkenyl, cycloalkyl, cycloalkenyl, substituted or unsubstituted aryl, substituted or unsubstituted heteroaryl, substituted or unsubstituted heteroalicyclyl, —CN, —C(═Z)R₄, —C(═Z)OR₄, —C(═Z)NR₄R₄ₐ, —N(R₄)—C(═Z)R₄ₐ, —N(R₄)—C(═Z)NR_{4b}R₄ₐ, —OC(═Z)R₄, and —SR₄;
R₁ₐ is selected from the group consisting of hydrogen, alkyl, alkenyl, alkynyl, aryl, halogen, hydroxyl, nitro, amino, sulfonyl, perhaloalkyl, —OR₄, —NR₄R₄ₐ, —CN, —C(═Z)R₄, —C(═Z)OR₄, —C(═Z)NR₄R₄ₐ;
R₂ is selected from the group consisting of hydrogen, alkyl, alkenyl, cycloalkyl, cycloalkenyl, substituted or unsubstituted aryl and unsubstituted or substituted heteroaryl; and,
R₃ is selected from the group consisting of hydrogen, alkyl, alkenyl, cycloalkyl, cycloalkenyl, substituted or unsubstituted aryl, substituted or unsubstituted heteroaryl, substituted or unsubstituted heteroalicyclyl, hydroxy, perhaloalkyl, —OR₄, —NR₄R₄ₐ, —NR₄NR₄ₐR_{4b}, —NR₄N═CR₄ₐR_{4b}, —N(R₄)C(R₄ₐ)═NR_{4b}, —N(R₄)—C(═Z)R₄ₐ, —N(R₄)—C(═Z)NR_{4b}R₄ₐ, and —SR₄, wherein:
Z is oxygen or sulfur; and,
R₄, R₄ₐ and R_{4b} are independently selected from the group consisting of: hydrogen, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroaryl, substituted or unsubstituted heteroalicyclyl.

2. The compound of claim 1, wherein:
R₃ is selected from the group consisting of —CH₂R₅, —OR₅ₐ and —NR_{5b}R_{5c}, wherein:
R₅ is selected from the group consisting of hydrogen; alkyl, alkenyl, cycloalkyl, cycloalkenyl, substituted or unsubstituted aryl, substituted or unsubstituted heteroaryl, hydroxy, nitro, amino, halogen, sulfonyl, perhaloalkyl, —OR₄, —NR₄R₄ₐ, —N═CR₄R₄ₐ, —C(R₄)═NR₄ₐ, —CN, —C(═Z)R₄, —C(═Z)OR₄, —C(═Z)NR₄R₄ₐ, —N(R₄)—C(═Z)R₄ₐ, —N(R₄)—C(═Z)NR₄ₐR_{4b}, —OC(═Z)R₄, and —SR₄;
R₅ₐ is selected from the group consisting of hydrogen; alkyl, alkenyl, cycloalkyl, cycloalkenyl, substituted or unsubstituted aryl, substituted or unsubstituted heteroaryl, and,
R_{5b} and R_{5c}, is selected from the group consisting of hydrogen; alkyl, alkenyl, cycloalkyl, cycloalkenyl, substituted or unsubstituted aryl, substituted or unsubstituted heteroaryl, hydroxy, amino, sulphonate, perhaloalkyl, —OR₄, —NR₄R₄ₐ, —N═CR₄R₄ₐ, —C(R₄)═NR₄ₐ, —CN, —C(═Z)R₄, —C(═Z)OR₄, —C(═Z)NR₄R₄ₐ, —N(R₄)—C(═Z)R₄ₐ, —N(R₄)—C(═Z)NR₄ₐR_{4b}, —OC(═Z)R₄, and —SR₄.

3. The compound of claim 1, wherein:
R₃ is selected from the group consisting of 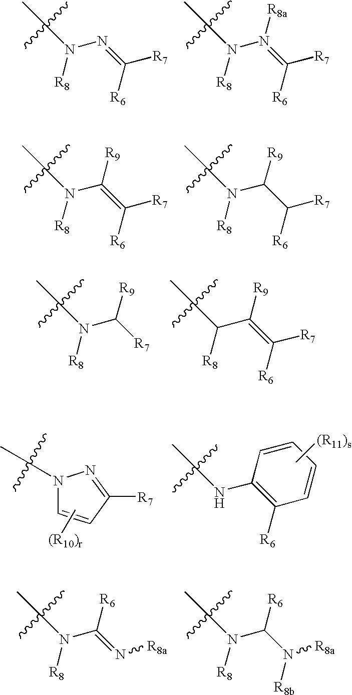
wherein;
R₆, R₇, and R₉ are independently selected from the group consisting of hydrogen, alkyl, alkenyl, cycloalkyl, cycloalkenyl, substituted or unsubstituted aryl, substituted or unsubstituted heteroaryl, substituted or unsubstituted heteroalicyclyl, hydroxy, nitro, amino, halogen, sulfonate, perhaloalkyl, —OR₄, —NR₄R₄ₐ, —CN, —C(═Z)R₄, —C(═Z)OR₄, —C(═Z)NR₄R₄ₐ, —N(R₄)—C(═Z)R₄ₐ, N(R₄)—C(═Z)NR₄ₐR_{4b}, —OC(═Z)R₄, and —SR₄;
R₈, R₈ₐ and R_{8b} are independently selected from the group consisting of hydrogen, alkyl, alkenyl, cycloalkyl, cycloalkenyl, substituted or unsubstituted aryl; substituted or unsubstituted heteroaryl, substituted or unsubstituted heteroalicyclyl, hydroxy, nitro, amino, halogen, sulfonyl, perhaloalkyl, —OR₄, —NR₄R₄ₐ, —CN, —C(═Z)R₄, —C(═Z)OR₄, —C(═Z)NR₄R₄ₐ, —N(R₄)—C(═Z)R₄ₐ, —N(R₄)—C(═Z)NR₄ₐR_{4b}, —OC(═Z)R₄, and —SR₄;
R₁₀ and R₁₁ are independently selected from the group consisting of hydrogen, alkyl, alkenyl, cycloalkyl, cycloalkenyl, substituted or unsubstituted aryl; substituted or unsubstituted heteroaryl, substituted or unsubstituted heteroalicyclyl, hydroxy, nitro, amino, halogen, sulfonyl, perhaloalkyl, —OR₄, —NR₄R₄ₐ, —CN, —C(═Z)R₄, —C(═Z)OR₄, —C(═Z)NR₄R₄ₐ, —(R₄)—C(═Z)R₄ₐ, —N(R₄)—C(═Z)NR₄ₐR_{4b}, —OC(═Z)R₄, and —SR₄;
r is 0, 1 or 2; and
s is 0, 1, 2, 3 or 4.

4. The compound of claim 1, wherein:
R₁ is selected from the group consisting of substituted or unsubstituted aryl, substituted or unsubstituted heteroaryl, wherein if substituted, the substituent is one or more independently selected from the group consisting of alkyl, alkenyl, cycloalkyl, cycloalkenyl, substituted or unsubstituted aryl, substituted or unsubstituted heteroaryl, substituted or unsubstituted heteroalicyclyl, hydroxy, nitro, amino, halogen, sulfonate, perhaloalkyl, —OR₄, —NR₄R₄ₐ, —CN, —C(═Z)R₄, —C(═Z)OR₄, —C(═Z)NR₄R₄ₐ, —N(R₄)—C(═Z)R₄ₐ, —N(R_{4b})—C(═Z)NR₄R₄ₐ, —OC(═Z)R₄, and —SR₄.

5. The compound of claim 1, wherein:
R₃ is selected from the group consisting of: 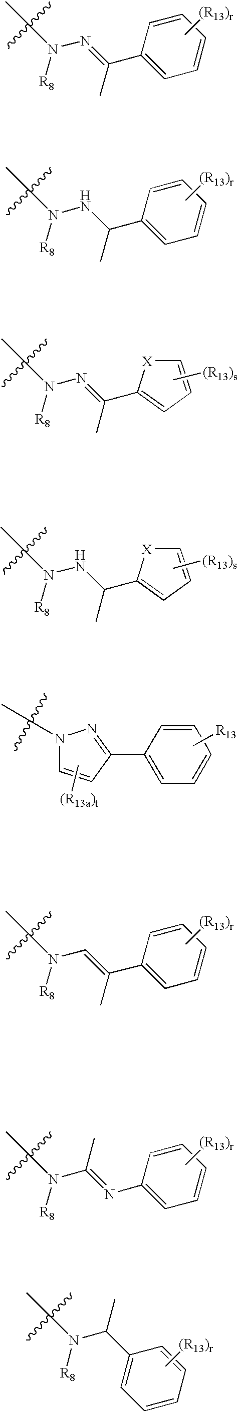
wherein:
R₈ is hydrogen or alkyl;
X is NH, O or S;
each R₁₃ is independently selected from the group consisting of hydrogen, alkyl, alkenyl, alkynyl, hydroxy, nitro, amino, halogen, sulfonyl, perhaloalkyl, —OR₄, —NR₄R₄ₐ, —CN, —C(═Z)R₄, —C(═Z)OR₄, —C(═Z)NR₄R₄ₐ, —N(R₄)—C(═Z)R₄ₐ, —N(R_{4b})—C(═Z)NR₄R₄ₐ, —OC(═Z)R₄, and —SR4;
r is 0, 1, 2, 3,4 or 5;
s is 0, 1, 2, 3 or 4; and,
t is 0, 1 or 2.

6. A method of treating or preventing a disease or disorder related to abnormal PAR2 activity, comprising administering a therapeutically effective amount of a compound of claim 1 to a patient in need thereof.

7. The method of claim 6, wherein the disease or disorder is selected from the group consisting of:
acute or chronic pain;
acute or chronic inflammation;
diseases or disorder of the pulmonary system;
diseases or disorders of the gastrointestinal system;
diseases or disorders of the musculoskeletal system;
diseases or disorders of the central nervous system;
diseases or disorders of the cardiovascular system;
disease or disorders of the renal system;
diseases or disorders of the hepatic system;
diseases of disorders of the eye;
diseases or disorders of the skin;
diseases or disorders of the prostrate;
diseases or disorders of the pancreas;
Sjogren's syndrome; and,
dry mouth.

8. The method of claim 7, wherein the disease or disorder of the pulmonary system is selected from the group consisting of asthma, chronic obstructive pulmonary disease, lung cancer and pneumonitis.

9. The method of claim 7, wherein the disease or disorder of the gastrointestinal system is selected from the group consisting of gastric ulcers, colitis, inflammatory bowel syndrome, Crohn's disease, gastric and intestinal motility, colon cancer, cancer of the stomach, and cancer of the intestine.

10. The method of claim 7, wherein the disease or disorder of the musculoskeletal system is selected from the group consisting of rheumatoid arthritis, osteoporosis and Paget's disease.

11. The method of claim 7, wherein the disease or disorder of the central nervous system is selected from the group consisting of Alzheimer's disease, encephalitis, meningitis, ischemia and stroke.

12. The method of claim 7, wherein the disease or disorder of the cardiovascular system is selected from the group consisting of hypertension, atherosclerosis, angina, congestive heart failure, myocarditus and cardiac ischemia.

13. The method of claim 7, wherein the disease or disorder of the renal system is selected from the group consisting of glomerular kidney disease, kidney cancer and renal failure.

14. The method of claim 7, wherein the disease or disorder of the hepatic system is selected from the group consisting hepatitis and liver cancer.

15. The method of claim 7, wherein the disease or disorder of the eye is selected from the group consisting of glaucoma, retinitis pigmentosa, cataracts, macular degeneration and dry eye.

16. The method of claim 7, wherein the disease or disorder of the skin is selected from the group consisting of dermatitis, psoriasis, pruritis, dermatitis, eczema, seborrhea, wounds, and melanoma.

17. The method of claim 7, wherein the disease or disorder of the pancreatic system is selected from the group consisting of pancreatitus, pancreatic cancer and diabetes.

18. The method of claim 7, wherein the disease or disorder is dry mouth.

19. The method of claim 7, wherein the the disease or disorder is Sjogren's syndrome.

20. The method of claim 7, wherein the disease or disorder is acute or chronic pain.

21. The method of claim 7, wherein the disease or disorder is acute or chronic inflammation.

22. The method of claim 7, wherein the disease of disorder of the prostatic system is selected from the group consisting of benign prostatic hyperplasia and prostatic cancer.

23. The method of claim 7, wherein the disease or disorder of the pancreatic system is selected from the group consisting of pancreatitis, diabetes and pancreatic cancer.

24. The method of claim 1 wherein the compound is selected from the group consisting of:
2Oxo-4-phenyl-3-pyrrolidinecarboxylic acid [1-(3-bromo-phenyl)-(E/Z)-ethylidene]-hydrazide (3)
2-Oxo-4-phenyl-3-pyrrolidinecarboxylic acid [1-(5-bromo-thiophen-2-yl)-(E/Z)-ethylidene]-hydrazide (4)
2-Oxo-4-phenyl-3-pyrrolidinecarboxyqic acid [1-(thiophen-2-yl)-(E/Z)-ethylidene]-hydrazide (5)
2-Oxo-4-phenyl-3-pyrrolidinecarboxylic acid [1-(4-bromo-thiophen-2-yl)-(E/Z)-ethylidene]-hydrazide (6)
2-Oxo-4-phenyl-3-pyrrolidinecarboxylic acid [1-(5-bromo-pyridine-3-yl)-(E/Z)-ethylidene]-hydrazide (7)
2-Oxo-4-phenyl-3-pyrrolidinecarboxylic acid [1-(4-bromo-phenyl)-(E/Z)-ethylidene]-hydrazide (8)
2-Oxo-4-phenyl-3-pyrrolidinecarboxylic acid [1-(2-bromo-phenyl)-(E/Z)-ethylidene]-hydrazide (9)
2-Oxo-4-phenyl-3-pyrrolidinecarboxylic acid [1-(3-methoxy-phenyl)-(E/Z)-ethylidene]-hydrazide (10)
2-Oxo-4-phenyl-3-pyrrolidinecarboxylic acid [1-(toluen-3-yl)-(E/Z)-ethylidene]-hydrazide (11)
2-Oxo-4-phenyl-3-pyrrolidinecarboxylic acid [1-(3-trifluoromethyl-phenyl)-(E/Z)-ethylidene]-hydrazide (12)
2-Oxo-4-phenyl-3-pyrrolidinecarboxylic acid N′-[1-(3-bromo-phenyl)-(E/Z)-ethyl]-hydrazide (13)
2-oxo-4-phenyl-3-pyrrolidinecarboxylic acid 2-[(E/Z)-1-(3,5-dimethylphenyl)ethylidene]hydrazide (14)
2-oxo-4-phenyl-3-pyrrolidinecarboxylic acid 2-[(1 E/Z)-7-bromo-2,3-dihydro-4-methyl-1H-inden-1-ylidene]hydrazide (15)
2-oxo-4-phenyl-3-pyrrolidinecarboxylic acid 2-[(1 E/Z)-5-(acetylamino)-2,3-dihydro-1H-inden-1-ylidene]hydrazide (16)
2-oxo-4-phenyl-3-pyrrolidinecarboxylic acid 2-[(E/Z)-1-(5-carboxy-2-thienyl)ethylidene]hydrazide (17)
2-oxo-4-phenyl-3-pyrrolidinecarboxylic acid 2-[(E/Z)-1-(4-chlorophenyl)-3-(dimethylamino)propylidene]hydrazide (18)
2-oxo-4-phenyl-3-pyrrolidinecarboxylic acid 2-[(E/Z)-1-(1H-pyrrol-2-yl)ethylidene]hydrazide (19)
2-oxo-N′-[(E/Z)-1-(3-oxo-3,4-dihydro-2H-1,4-benzoxazin-6-yl)ethylidene]-4-phenyl-3-pyrrolidinecarbohydrazide (20)
N′-[(4 E/Z)-2,3-dihydro-4H-chromen-4-ylidene]-2-oxo-4-phenyl-3-pyrrolidinecarbohydrazide (21)
2-oxo-4-phenyl-3-pyrrolidinecarboxylic acid 2-[(E/Z)-1-(3-fluorophenyl)ethylidene]hydrazide (22)
2-oxo-4-phenyl-3-pyrrolidinecarboxylic acid 2-[(E/Z)-1-(3-chlorophenyl)ethylidene]hydrazide (23)
2-oxo-4-phenyl-3-pyrrolidinecarboxylic acid 2-[(E/Z)-1-(3-cyanophenyl)ethylidene]hydrazide (24)
2-oxo-4-phenyl-3-pyrrolidinecarboxylic acid 2-[(E/Z)-1-(3-hydroxyphenyl)ethylidene]hydrazide (25)
2-oxo-4-phenyl-3-pyrrolidinecarboxylic acid 2-[(E/Z)-1-(3,5-dimethoxyphenyl)ethylidene]hydrazide (26)
2-oxo-4-phenyl-3-pyrrolidinecarboxylic acid 2-[(E/Z)-1-[3,5-bis(trifluoromethyl)phenyl]ethylidene]hydrazide (27)
2-oxo-4-phenyl-3-pyrrolidinecarboxylic acid 2-[(E/Z)-1-(3,5-difluorophenyl)ethylidene]hydrazide (28)
2-oxo-4-phenyl-3-pyrrolidinecarboxylic acid 2-[(E/Z)-1-(3,4-dimethylphenyl)ethylidene]hydrazide (29)
2-oxo-4-phenyl-3-pyrrolidinecarboxylic acid 2-[(E/Z)-1-(1,3-benzodioxol-5-yl)ethylidene]hydrazide (30)
2-oxo-4-phenyl-3-pyrrolidinecarboxylic acid 2-[(E/Z)-1-(2,3-dihydro-1,4-benzodioxin-6-yl)ethylidene]hydrazide (31)
2-oxo-4-phenyl-3-pyrrolidinecarboxylic acid 2-[(E/Z)-1-(2,3-dihydro-1,4-benzodioxin-6-yl)ethylidene]hydrazide (32)
2-oxo-4-phenyl-3-pyrrolidinecarboxylic acid 2-[(E/Z)-1-(2,3-dihydro-2-oxo-6-benzothiazolyl)ethylidene]hydrazide (33)
2-oxo-4-phenyl-3-pyrrolidinecarboxylic acid 2-[(E/Z)-1-(2,5-dichloro-3-thienyl)ethylidene]hydrazide (34)
2-oxo-4-phenyl-3-pyrrolidinecarboxylic acid 2-[(E/Z)-1-(2,5-dimethyl-3-thienyl)ethylidene]hydrazide (35)
2-oxo-4-phenyl-3-pyrrolidinecarboxylic acid 2-[(E/Z)-1-(2,5-dimethyl-3-furanyl)ethylidene]hydrazide (36)
2-oxo-4-phenyl-3-pyrrolidinecarboxylic acid 2-[(E/Z)-1-(2,5-dimethyl-3-furanyl)ethylidene]hydrazide (37)
2-oxo-4-phenyl-3-pyrrolidinecarboxylic acid 2-[(E/Z)-1-(5-chloro-2-thienyl)ethylidene]hydrazide (38)
2-oxo-4-phenyl-3-pyrrolidinecarboxylic acid 2-[(E/Z)-1-(5-methyl-2-thienyl)ethylidene]hydrazide (39)
2-oxo-4-phenyl-3-pyrrolidinecarboxylic acid 2-[(E/Z)-1-(5-cyano-2-thienyl)ethylidene]hydrazide (40)
2-oxo-4-phenyl-3-pyrrolidinecarboxylic acid 2-[(E/Z)-1-(5-bromo-2-hydroxyphenyl)ethylidene]hydrazide (41)
2-oxo-4-phenyl-3-pyrrolidinecarboxylic acid 2-[(E/Z)-1-(4-methyl-2-thienyl)ethylidene]hydrazide (42)
2-oxo-4-phenyl-3-pyrrolidinecarboxylic acid 2-[(1 E/Z)-2,3-dihydro-5-methoxy-1H-inden-1-ylidene]hydrazide (43)
2-oxo-4-phenyl-3-pyrrolidinecarboxylic acid 2-[(1 E/Z)-2,3-dihydro-5,6-dimethoxy-1H-inden-1-ylidene]hydrazide (44)
2-oxo-4-phenyl-3-pyrrolidinecarboxylic acid 2-[(1 E/Z)-5-fluoro-2,3-dihydro-1H-inden-1-ylidene]hydrazide (45)
2-oxo-4-phenyl-3-pyrrolidinecarboxylic acid 2-[(1 E/Z)-2,3-dihydro-6-methoxy-1H-inden-1-ylidene]hydrazide (46)
2-oxo-4-phenyl-3-pyrrolidinecarboxylic acid 2-[(1 E/Z)-5-bromo-2,3-dihydro-1H-inden-1-ylidene]hydrazide (47)
2-oxo-4-phenyl-3-pyrrolidinecarboxylic acid 2-[(1 E/Z)-2,3-dihydro-6-methyl-1H-inden-1-ylidene]hydrazide (48)
2-oxo-4-phenyl-3-pyrrolidinecarboxylic acid 2-[(1 E/Z)-2,3-dihydro-4,5-dimethoxy-1H-inden-1-ylidene]hydrazide (49)
2-oxo-4-phenyl-3-pyrrolidinecarboxylic acid 2-[(E/Z)-1-(2,4-dimethoxyphenyl)ethylidene]hydrazide (50)
2-oxo-4-phenyl-3-pyrrolidinecarboxylic acid 2-[(E/Z)-1-(2,6-difluorophenyl)ethylidene]hydrazide (51)
2-oxo-4-phenyl-3-pyrrolidinecarboxylic acid 2-[(E/Z)-1-(3-fluoro-4-methoxyphenyl)ethylidene]hydrazide (52)
2-oxo-4-phenyl-3-pyrrolidinecarboxylic acid 2-[(E/Z)-1-(3,4-difluorophenyl)ethylidene]hydrazide (53)
2-oxo-4-phenyl-3-pyrrolidinecarboxylic acid 2-[(4 E/Z)-6-fluoro-2,3-dihydro-4H-1-benzothiopyran-4-ylidene]hydrazide (54)
2-oxo-4-phenyl-3-pyrrolidinecarboxylic acid 2-[(E/Z)-1-(2,3-difluorophenyl)ethylidene]hydrazide (55)
2-oxo-4-phenyl-3-pyrrolidinecarboxylic acid 2-[(4 E/Z)-2,3-dihydro-6-methyl-4H-1-benzopyran-4-ylidene]hydrazide (56)
2-oxo-4-phenyl-3-pyrrolidinecarboxylic acid 2-[(E/Z)-1-(2,3,4,5,6-pentafluorophenyl)ethylidene]hydrazide (57)
2-oxo-4-phenyl-3-pyrrolidinecarboxylic acid 2-[(E/Z)-1-[3-(acetylamino)phenyl]ethylidene]hydrazide (58)
2-oxo-4-phenyl-3-pyrrolidinecarboxylic acid 2-[(E/Z)-1-(1-methyl-1H-pyrrol-3-yl)ethylidene]hydrazide (59)
2-oxo-4-phenyl-3-pyrrolidinecarboxylic acid 2-[(E/Z)-1-(2,4-dimethyl-1H-pyrrol-3-yl)ethylidene]hydrazide (60)
2-oxo-4-phenyl-3-pyrrolidinecarboxylic acid 2-[(E/Z)-1-(1-methyl-1H-pyrrol-2-yl)ethylidene]hydrazide (61)
2-oxo-4-phenyl-3-pyrrolidinecarboxylic acid 2-[(E/Z)-1-(3-thienyl)ethylidene]hydrazide (62)
2-oxo-4-phenyl-3-pyrrolidinecarboxylic acid 2-[(1 E/Z)-3,4-dihydro-7-nitronaphthalenylidene]hydrazide (63)
2-oxo-4-phenyl-3-pyrrolidinecarboxylic acid 2-[(1 E/Z)-6-bromo-2,3-dihydro-1H-inden-1-ylidene]hydrazide (64)
2-oxo-4-phenyl-3-pyrrolidinecarboxylic acid 2-[(1 E/Z)-5-chloro-2,3-dihydro-6-methoxy-1H-inden-1-ylidene]hydrazide (65)

25. A method for the treatment or prevention of a disease or disorder related to abnormal PAR2 activity comprising administering to a patient in need thereof a compound of claim 24.
